(19) United States
(12) Patent Application Publication
(10) Pub. No.: US 2025/0312527 A1
(43) Pub. Date: Oct. 9, 2025

(54) FLUID GUIDE SYSTEM USABLE FOR DRUG HANDLING, E.G. FOR RECONSTITUTION, CORRESPONDING MEDICAL DEVICE, METHOD AND COMPUTER RELATED ITEMS

(71) Applicant: GENZYME CORPORATION, Cambridge, MA (US)

(72) Inventors: Craig Nelson, Melbourne (GB); Alexander Hee-Hanson, Melbourne (GB); Tom Lever, Melbourne (GB); Haiming Wu, Cambridge, MA (US)

(73) Assignee: GENZYME CORPORATION, Cambridge, MA (US)

(21) Appl. No.: 18/628,338

(22) Filed: Apr. 5, 2024

(51) Int. Cl. A61M 5/14; A61M 5/162; A61M 5/172

(52) U.S. Cl. CPC A61M 5/1409; A61M 5/162; A61M 5/172

(57) ABSTRACT

Disclosed is a fluid guide system (FGS) usable for drug (D) handling, comprising:
At least one fluid port (FP) configured to dispense a fluid from the fluid guide system (FGS), and
At least two drug port (DP), each being configured to receive a fluid comprising at least one drug (D) from at least one drug container (DC),
   wherein each of the at least two drug ports (DC) is coupleable or coupled to at least one drug container (DC) such that a plurality of drug containers (DC) can be or is jointly mechanically coupled to the fluid guide system (FGS).

The disclosure relates to a medical device usable for drug handling, e.g. for reconstitution. Single vial reconstitution may be possible using a dedicated medical device.

There is an ever increasing demand for drug handling devices, e.g. for devices that enable non HCP (health care personnel) but also HCP to handle drugs prior to injection or prior to infusion. The demand is driven by the fact that more and more drugs are lyophilized, e.g. biomedical drugs, or other drugs, e.g. insulin. In short, lyophilization is a freeze drying process in which a liquid drug goes through at least one cycle of freezing and/or sublimation in a vacuum to become solid, e.g. at sub-freezing temperatures, e.g. at temperatures below the freezing point of the liquid drug. After the drugs have first been frozen, a vacuum may reduce the pressure until sublimation takes place, e.g. the transition of a substance directly from the solid state to the gas state, without passing through the liquid state. The advantage is that the lyophilized drugs may be transported more easily and may be stored for longer times compared to e.g. liquid drug formulations. Thus, the lyophilization industry is a steady growing industry. Correspondingly, the demand for reconstitution devices increases steadily too.

Associated with a lyophilized drug, there may be the challenge to prepare them prior to use when reconstitution is required. This would be even more so when multiple vials are needed, or when a variable amount of drug is needed, e.g. such as weight based dosing. The current invention may address at least one of these issues or all of them.

However, similar devices may be used for other drug handling, e.g. for mixing liquid and/or lyophilized drugs that are personalized, e.g. preparing mixtures of drugs for treating cancer or other diseases.

### SUMMARY

A fluid guide system usable for drug handling, comprising:
At least one fluid port configured to dispense a fluid from the fluid guide system, and/or
At least two drug ports, each drug port being configured to receive a fluid comprising at least one drug from at least one drug container,
   wherein each of the at least two drug ports may be coupleable or coupled to at least one drug container such that a plurality of drug containers can be or is jointly mechanically coupled to the fluid guide system.

A medical device for drug handling, comprising:
A case, and/or
A support portion arranged within the case or on the case and configured to support a removable fluid guide system, preferably a fluid guide system as mentioned above, and at least one electronic control unit,
   wherein the fluid guide system may comprise at least two drug ports configured to be connected to a respective one of at least two drug containers comprising at least one drug, and
   wherein the at least one electronic control unit may be configured to control the operation of the medical device during drug handling using the at least one drug.

A method for drug handling, comprising:
Preferably, using a fluid guide system that comprises a fluid channel system and at least two drug ports, each drug port being configured to receive a fluid comprising at least one drug from at least one drug container, and/or
Preferably, using a medical device comprising a supporting portion for the fluid guide system, and/or
Preferably, mechanically coupling the at least two drug containers to the at least two drug ports jointly during handling of the at least drug, and/or
Preferably, applying the fluid guide system to the supporting portion of the device before or after coupling, and/or
Performing at least one drug handling step automatically using the at least one medical device e.g. as mentioned above when a fluid guide system is applied to the supporting portion of the device, e.g. the fluid guide system as mentioned above.

It is an object of the disclosed embodiments to provide a fluid guide system, e.g. a cartridge system or a flexible carrier system. The fluid guide system should be preferably be a simple system and/or a low cost system. Further, the fluid guide system should preferably enable usage of at least two drug containers for handling of the drug(s), e.g. reconstitution and/or mixing of drugs. Preferably, the system should allow fast drug handling, e.g. fast reconstitution. Further, the system should be able to provide a low and/or minimal contamination and/or sterility loss risk. Additionally or optionally, the system should be a safe system with regard to the health of the patient(s), e.g. preventing or mitigating cross contamination and/or contamination, e.g. loss of sterility. Furthermore, a corresponding medical device, a corresponding method and corresponding computer related items shall be provided.

This object is solved by the fluid guide system according to claim 1, by the medical device according to the independent device claim, by the method according the independent method claim and by the computer related issues. Further embodiments are mentioned in the dependent claims.

According to an aspect, a fluid guide system usable for drug handling is provided, comprising:
At least one fluid port configured to dispense a fluid from the fluid guide system, and/or
At least two drug ports, each drug port being configured to receive a fluid comprising at least one drug from at least one drug container.

According to an embodiment, each of the at least two drug ports may be coupleable or coupled to at least one drug container such that a plurality of drug containers can be or is jointly mechanically coupled to the fluid guide system. Jointly may mean that there is a state in which the at least two drug containers are mechanically coupled simultaneously or at the same time to the fluid guide system,

The number of drug containers may be chosen appropriately for the envisaged application scenario. To give only a non-restricting example, the number of drug containers may be in the range of 2 to 100, in the range of 2 to 50, in the range of 3 to 40 or in the range of 4 to 30. Repeated loading may be used in order to reduce the number of drug ports of the device, e.g. for preparing one dosage for a specific patient.

Moreover, exemplarily, all drug containers may have the same shape, e.g. vials of a specific type and/or fill volume. This may reduce the production costs of the drug ports. However, alternatively or additionally, several types of drug containers and/or of drug ports may be used, e.g. at least two types, at least three types, etc.

Further, the same drug may be comprised in all drug containers. Alternatively, in at least one of the drug containers, there may be a drug that is different from the drug comprised in at least one other drug container. Thus, exemplarily, at least one type of drug, at least two types of drug, or at least three types of drugs may be used, e.g. for preparing one dosage for a specific patient.

However, filling of the at least two drug containers may occur sequentially or simultaneously. Similarly, emptying of the at least two drug containers may occur sequentially or simultaneously.

The fluid guide system may be a removable guide system which may be removable from a medical device used for drug handling without e.g. using a tool and/or without destroying the medical device. The fluid guide system may be disposable, i.e. consumable. “Consumable” may mean that it is used only once. Thus, no cleaning of the medical device may be necessary after each use. No cross contamination between several drug handlings may be possible, etc. Preferably, the fluid guide system comprises mainly a low cost material, e.g. a plastic material.

The drug port, may be a drug container coupling port, e.g. a vial coupling port. The drug port may comprise e.g. a fluid port function and/or a mechanical coupling function.

Thus, a fluid guide system is provided to which at least drug containers may be mechanically coupled jointly. This opens an option to use only one of the drug ports of to use both drug ports. Moreover, if the at least two drug containers are coupled jointly to the fluid guide system, an automated drug handling process may be used to handle the drug comprised in each of the at least two drug containers. Other technical effects are apparent from the further description, e.g. fast drug handling after all drug containers are connected to respective drug ports.

According to an embodiment, the fluid guide system may comprise:
A fluid channel system, and/or
A carrier configured to carry the fluid channel system.

Each of the at least two drug ports may comprise a respective fluid transmitting portion that may be configured to transmit fluid coming from the respective drug port of the at least two drug ports, preferably to the fluid guide system. Alternatively and/or additionally, e.g. in a reconstitution process, the respective fluid transmitting portion may be configured to transmit fluid coming from the fluid guide system to the respective drug port of the at least two drug ports.

The fluid channel system may comprise the at least one fluid port and the fluid portions of the at least two drug ports. The fluid channel system may be configured to be supported by a medical device, e.g. to be received within a receiving space, preferably within a closable receiving space. Alternatively, an open support portion may be used, e.g. comprising a support surface.

The carrier may be a rigid carrier or a flexible carrier. Gravity may not deform the rigid carrier. On the contrary, gravity may deform the flexible carrier, e.g. if hold only one edge. The fluid channel system may be a fluid guide component, e.g. an integral component of the fluid guide system, e.g. a molded component or a heat sealed component. Thus, the fluid guide system may be a compact system which is easily to transport and/or which may be easy inserted into a receiving space or the support portion of a medical device.

According to an embodiment, the carrier may extend laterally via an area comprising at least two drug ports, at least three drug ports, etc., preferably comprising all drug ports of the fluid guide system.

The carrier may be non-separable, e.g. in a lateral direction (width and/or length direction), especially not without using a special tool (e.g. saw) and/or without destruction. The carrier may be integrally formed, i.e. not formed by coupling at least two modules, e.g. not laterally and/or not staggering of modules one upon the other. The carrier may be free of mechanical coupling elements as may be used between several modules which are coupled together. This may result in a simple and/or reliable carrier. Thus, the carrier may comprise at least one plate or foil extending laterally to all drug ports, especially an integrally formed plate of foil. There may be only one such plate or foil. Alternatively, at least two such plates/foils or exactly two plates or foils may be arranged relative to each other within the fluid guide system, e.g. parallel or essentially parallel relative to each other.

The drug ports may be configured to receive only a drug container, especially only one drug container, e.g. a vial, especially without further coupling modules arranged between the respective drug container, e.g. a vial and the respective drug port. Thus, the fluid guide system may be simple and/or reliable.

According to an embodiment, at least one of the following may be implemented:

Variant a) A width and a length and/or a height of the fluid guide system may be adapted to a width and a length and/or a height of a retaining space of a medical device for retaining the fluid guide system. Preferably the retaining space may be a coverable retaining space that is configured to be covered by a cover, e.g. by a pivotable cover which is pivotable about a hinge.

A difference between the length (e.g. maximal length) of fluid guide system and the length (e.g. maximal length) of the retaining space may be at most 5% (percent) of length of fluid guide system, e.g. less than 10 mm (millimeter) or less than 5 mm or less than 2 mm.

A difference between the width (e.g. maximal width) of fluid guide system and the width (e.g. maximal width) of the retaining space may be at most 5% of the width of the fluid guide system, e.g. less than 10 mm (millimeter) or less than 5 mm or less than 2 mm.

A difference between the height (e.g. maximal height) of the fluid guide system and height (e.g. maximal height) of the retaining space may be at most 5% of height of fluid guide system, e.g. less than 10 mm (millimeter) or less than 5 mm or less than 2 mm.

Thus the retaining space may complement the outer contour of the fluid guide system. This may allow a small device, e.g. comprising a small construction space. The retaining space may be covered by a cover, e.g. in order to guarantee that the mixing/reconstitution process is performed properly without external interruptions.

Variant b) At least one hole or other recess may be provided within the fluid guide system for the arrangement of a pump or of at least a part of the pump on the fluid guide system. Preferably, a positive displacement pump may be used, e.g. a peristaltic pump. Further preferably, the pump may be part of a medical device that may comprise a retaining space for retaining the fluid guide system. Thus, the pump may be part of the reusable system while the fluid guide system may be part of the consumable and/or disposable system.

Variant c) An inlet port of the fluid guide system may be configured to be connected or may be connected to a flexible inlet tube. An outlet port of the fluid guide system may be configured to be connected or may be connected to a flexible outlet tube. Preferably, the geometrical arrangement of the inlet port relative to the outlet port may correspond to the geometrical arrangement of at least one hole or of at least two holes providing access to a retaining space of a medical device (e.g. machine). The retaining space may be configured to retain the fluid guide system. Further preferably, the at least one hole or the at least two holes may be configured to allow passage of a portion of the inlet tube and of a portion of the outlet tube.

“Correspond to” may mean “to be equal to”, e.g. in order to allow exact or almost exact geometrical mapping. The at least one hole may allow access to the retaining space. The at least one hole may be arranged within a cover of retaining space. Thus, the machine may not come into contact, especially into direct contact with the fluid(s). Therefore, cleaning of the machine may not be necessary between two operating phases using two disposable fluid guide systems (e.g. cartridges or flexible fluid guide systems). The second operating phase may follow the first operating phase without at least one intermediate operation phase. Alternatively, there may be at least one intermediate operating phase between the two operating phases, e.g. at least 5, at least 50, or at least 100 intermediate operating phases.

According to an embodiment, the carrier may comprise a rigid cartridge. The rigid cartridge may comprise a rigid case, e.g. an outer case. The rigid case may comprise at least two openings configured to allow access to the at least two drug ports in order to couple a respective one of the plurality of drug containers to a respective one of the at least two drug ports.

A rigid case may be robust against mechanical impact and/or environmental influences, e.g. against dust, heat, humidity. Injection molding and/or milling may be used to produce the rigid case, preferably exactly two or at least two parts of the rigid case.

At least a part of the fluid channel system may be formed integrally with the cartridge. Thus, the cartridge may comprise a lower part and an upper part. The upper part may comprise an upper portion of a respective channel. The lower part may comprise a lower portion of a respective channel. At least one part of the cartridge may be transparent or translucent, e.g. in order to enable inspection of the cartridge, preferably manual and/or automatic inspection.

The cartridge may be a flat cartridge, e.g. the length and the width may be much greater than the height, e.g. at least by factor 5 or factor 8 or factor 10 for the width and/or by at least by factor 5 or factor 8 or factor 10 or factor 15 for the length. Thus, less material may be necessary for the cartridge compared to bulky other forms, e.g. more like a bar or a cube.

The cartridge may comprise two outer parts, e.g. in the form of two shells or of one shell and one plate. This may facilitate production of the fluid guide system.

The cartridge may have a cuboid shape or an essentially cuboid shape, e.g. disregarding an extension “leg” and or irregularities due to a pump interface.

According to an embodiment, the carrier may comprise a flexible carrier portion. At least a part of the fluid channel system may be formed integrally with the flexible carrier by connecting at least two flexible sheet materials selectively thereby forming the fluid channel system.

The channels of the fluid guide system may be formed by connecting portions of two opposite sides of a flexible bag, e.g. of a plastic bag. However, two separate sheets, e.g. plastic sheets may be used as well. Heat sealing or gluing technologies may be used to produce the flexible bag. Thus, manufacturing may be easy.

At least on foil/sheet of the flexible carrier may be transparent or translucent, e.g. in order to enable inspection of the cartridge, preferably manual and/or automatic inspection.

The flexible carrier may be a flat carrier, e.g. the length and the width of the carrier may be much greater than the height, e.g. at least by factor 5 or factor 8 or factor 10 for the width and/or by at least by factor 5 or factor 8 or factor 10 or factor 15 for the length.

According to an embodiment, the fluid guide system may comprise at least one flow control portion. At least one of the at least two drug ports may be or the at least two drug ports may be fluidically connected or connectable to the at least one fluid port via the at least one flow control portion. The at least one flow control portion may be configured to allow a fluid flow through the at least one flow control portion in a flow-through operation mode of the at least one flow control portion and to prevent a fluid flow through the at least one flow control portion in a blocking operation mode of the at least one flow control portion.

The flow control portion may be switchable between the flow-through operation mode and the blocking operation mode. Switching may be possible repeatedly or only once, e.g. disruption of a foil within the cartridge or the flexible carrier. Switching may be possible by deforming a flexible portion, e.g. by compressing a tube and/or by actuating on a membrane/diaphragm. Opening of the flow control portion may be possible by releasing a pressure at a flexible portion or at the membrane/diaphragm.

At least two switching operations may be possible, e.g. from open to closed and from closed to open or vice versa. Alternatively or additionally, at least three switching operations may be possible, e.g. a) from open to closed and then from closed to open and thereafter further from open to close and/or b) from closed to open and then from open to close and thereafter from closed to open. Thus, the valve function may be different from a valve which is destroyed by switching from one state to another state, e.g. by tearing a foil of the valve. Thus, in contrast to permanent “switching” selective (non-permanent) switching may be possible.

Moreover, the switching operation may be an automatic switching operation that is e.g. different from a manual switching operation, e.g. during coupling of several fluidic modules.

The flow control portion may enable usage of at least two drug containers or of a plurality of drug containers, e.g. a number in the range of 2 to 50, in the range of 3 to 40 or in the range of 4 to 30. However, even if only one drug container is used, the flow control portion may allow to fill or to empty the drug container in an easy way. If several drug containers are used, one drug container or a part of the drug containers may be filled and/or emptied selectively compared to other drug containers that have to be used to complete the drug handling, e.g. reconstitution and/or drug mixing and/or drug accumulation, e.g. within a common further drug container. The further drug container may be e.g. an IV (intravenous) bag or a similar container.

The flow control portion may be part of a complete valve arranged within the fluid guide system, e.g. comprising a valve portion and an actuator. However, alternatively, the fluid guide system may only comprise the valve portion but not the valve actuator in order to enable low cost fluid guide systems, e.g. disposable or consumable fluid guide systems.

The fluid guide system may comprise a fluid channel system comprising at least one, at least two or at least three fluid channels. Bifurcations, valves, input ports and output ports may determine the end/beginning of a respective channel. There may be channels between which no fluid communication is possible, e.g. in a specific operation mode. However, in another operation mode a fluid communication may be possible between the same respective channels. This may allow to perform dedicated and/or sophisticated drug handling processes.

The drug handling/preparation may be e.g. a reconstitution and/or mixing of drugs, e.g. of the same drugs or of different drugs.

The fluid guide system may guide liquids or gases. According to a further embodiment, the fluid guide system may be a liquid guide system configured to guide liquids, e.g. to the at least one drug container and/or from the at least one drug container.

At least two drug ports may be used, e.g. in order to provide flexibility for drug handling, e.g. with regard to the amount of dose of one and the same drug and/or with regard to the components of a drug mixture.

There may be the following fluid paths within the fluid guide system, preferably within a channel system of the fluid guide system:
The fluid guide system may be configured to allow a fluid flow from at least one drug port through the at least one flow control portion to the at least one fluid port depending on the flow-through (e.g. open) operation mode of the at least one flow control portion,
The fluid guide system may be configured to prevent fluid flow out of at least one drug container to at least one fluid port or from at least one fluid port to at least one drug container depending on the blocking operation mode of the flow control portion.
At least one fluid port may be configured to receive a liquid from the external of the fluid guide system or the fluid guide system may comprise at least one fluid input port which is configured to receive a liquid from the external of the fluid guide system, e.g. from a medical device to which the fluid guide system may be coupled.
The drug port may be configured to transport fluid to a respective drug container, preferably fluid that is received via the at least one fluid port or via the at least one input port, preferably a reconstitution fluid, e.g. WFI (water for injection), etc.

The flow control portion may be one of the following:
A pinch valve or a pinch valve portion, e.g. a flexible tube or a flexible tube portion,
A diaphragm or a diaphragm valve,
Another type of valve or valve portion,
A tearable sheet portion, etc.

According to a further embodiment of the fluid guide system, the at least one drug portion may comprise at least one respective needle. The respective needle may be arranged movably relative to a case of the fluid guide system or the respective needle may be arranged within or on a flexible bag that forms the carrier of the fluid guide system.

This may allow automatic needle insertion of the at least one needle into the plug or other closure part of a drug container. Thus, needle sticks to body parts of a user may be prevented or reduced considerably.

An eccentric actuator or a linear actuator or another appropriate actuator may be used to move the needle during needle insertion.

Alternatively, the respective needle may be arranged stationary relative to an outer case of the fluid guide system. This may reduce production costs of the fluid guide system. Needle insertion may be done manually by a user or automatically during insertion of drug containers into the respective drug ports, e.g. by screwing, pushing, etc.

Further, alternatively, the respective needle may be arranged within or on a flexible bag that may form the carrier of the fluid guide system. Depending on the construction of a medical device, the needle may be stationary relative to a support surface for the flexible bag or the needle may be movable relative to a support surface for the flexible bag.

According to a further embodiment of the fluid guide system, e.g. of a cartridge system or of a flexible bag system, the at least one drug portion may comprise at least one mechanical element configured for fastening a respective one drug container of the at least one drug container or of the at least two drug containers. Preferably, at least one, several of or all of the following features may be used:
An internal screw thread configured to interact with a corresponding external screw thread of the at least one drug container, and/or
At least one hook configured to engage a recessed portion of the at least one drug container, and/or
At least one flexible portion adapted to provide a lateral force fit for holding of the at least one drug container at an opening end of the at least one drug container.

Thus, the drug ports of the fluid guide system may be adapted to the drug containers which are usable for drug handling. Snap connections may be used to prevent reuse of the fluid guide system, e.g. cartridge or bag and/or of the drug container(s).

Also a flexible (e.g. plastic) carrier may comprise the above mentioned fastening elements, e.g. at least one more rigid portion which may be e.g. heat sealed within the flexible carrier. Alternatively, a cover of the medical device may comprise the above mentioned fastening elements. Needles of the flexible carrier may then be moved to the fastening elements after placement of the drug containers. Alternatively, stationary needles may be used within the flexible carrier using needle insertion during insertion of the drug containers into the fastening elements.

According to a further embodiment, the fluid guide system may comprise at least two flow control portions of the at least one flow control portion. The fluid flow to each one of the at least two drug ports may be separately controllable, e.g. open (flow-through or blocking) via the operation mode of a respective one of the at least two flow control portions.

Alternatively, according to a further embodiment, there may be at least two drug ports fluidically connected to a common flow control portion, preferably without an intermediate flow control portions within the respective fluid paths from the common flow control portion to the respective flow control portion, e.g. a valve or a valve portion.

According to an embodiment, the at least two drug ports may comprise at least four drug ports. The at least four drug ports may be arranged in a matrix comprising at least two columns and at least two lines. Thus, the area occupied by the drug poets and/or drug containers may be small. However, according to another embodiment, alternatively other arrangements may be used, e.g. linear arrangement, circular, etc.

According to an embodiment, the fluid guide system may comprise at least one channel extending along a plurality of the at least two drug ports. At least two auxiliary channels may connect a respective drug port of the at least two drug ports fluidically with the channel. The respective auxiliary channel and the channel may form an angle within the range of 10 degrees to 80 degrees, in the range of 20 degrees to 70 degrees or in the range of 30 degrees to 60 degrees. Again, the area occupied by the overall structure (e.g. drug ports, main channels and/or auxiliary channels) may be small.

The auxiliary channels and the channel may form one half of a “fishbone” structure. A straight channel and/or straight auxiliary channels may be used.

The angle may be an acute angle, e.g. an angle of about 45 degrees or of 45 degrees. The at least one channel may be a main channel of matrix, e.g. extending in column direction.

According to an embodiment, the fluid guide system may comprise:
At least one output port configured to be connected or connectable to a further drug container, and/or
At least one input port configured to be connected or connectable to an auxiliary drug container.

The at least output port and the at least one input port may be both fluidically connectable or connected to at least one intermediate fluid channel of the fluid guide system. According to a variant a), the fluid guide system may comprise a pump interface unit selectively coupleable to the at least one input port and to the at least one output port. According to a variant b), the fluid guide system may comprise a pump selectively coupleable to the at least one input port and to the at least one output port.

This may allow a simple construction of the medical device and/or of the fluid guide system, e.g. a cartridge or a flexible carrier.

Preferably, the pump interface may comprise a flexible tube and at least one recess for retaining or for receiving a pump element. Thus, a peristaltic pump may be used. The driving parts of the peristaltic pump may be integral parts of the medical device but not of the fluid guide system, e.g. not of the cartridge. Thus, the fluid guide system may be a low cost part. Moreover, cleaning of the pump after each use may not be necessary as the reusable parts of the pump do not contact the liquids (or alternatively gases) during drug handling.

Other pumps may be used as well, e.g. other positive displacement pumps. Contrary to suction pumps (negative pressure pump), damage of the drugs during handling may be reduced or may occur less frequently. However, depending on the kind of drugs, usage of negative pressure pumps may also be considered.

According to an aspect, variant a), a set of fluid guide systems is provided, comprising at least one fluid guide system according to any one of the preceding embodiments and the following:
At least one fluid guide module which is configured to be coupled to at least one of the at least two drug ports via a common coupling port.

The at least one fluid guide module may comprise at least two further drug ports. The at least two further drug ports may be fluidically coupled or coupleable to the common coupling port of the at least one modular fluid guide system.

Preferably the module may be free of a flow control portion having at least two operation modes. Thus, the module may be simple and low cost.

The module of the modular system may allow reduction of waste, lowering of costs of the fluid guide system, etc. All fluid guide systems of the set according to variant a) may be configured to interact with the same medical device e.g. after placement of the at least one (basic) fluid guide system in the support portion/receiving space of the medical device. At least one of the modules may then be attached to the basic fluid guide system. Alternatively, the modules may be attached to the (basic) fluid guide system prior to insertion into the support portion/receiving space or prior to attachment of the fluid guide system to another support element of the medical device.

Additionally or alternatively, the at least one set of fluid guide systems, e.g. cartridge or flexible carrier may comprise in a variant b):
At least one further fluid guide system, wherein the further fluid guide system is configured to be applied to the same medical device as the fluid guide system according to any one of the preceding embodiments.

The further fluid guide system may comprise compared to the fluid guide system according to any one of the preceding aspects at least one, several of or all of:
A different fluid channel system, and/or
A different number of fluid control portions, and/or
A different number of drug ports, and/or
A different size, and/or
A different outer shape.

Thus, both fluid guide systems according to variant b) may have e.g. the same height, the same width, etc. Exemplary, an inlet port and an outlet port on both fluid guide systems may have the same relative positions to each other, e.g. corresponding to respective positions of ports on the medical device. The same may apply to the location of a pump or a pump interface unit.

Exemplary, the same reference point may be used on both fluid guide systems to align a coordinate system, e.g. a Cartesian coordinate system on both fluid guide system, e.g. a lower left corner. An inlet port of the first fluid guide system may have the same position relative to the first coordinate system as the inlet port of the second fluid guide system relative to the second coordinate system. The same may apply to an output port, to the fluid port, to a pump/pump interface, etc.

The set according to variant b) may also allow reduction of waste, lowering of costs of the fluid guide system, etc. All fluid guide systems of the set according to variant b) may be configured to interact with the same medical device after placement of e.g. only one fluid guide system of the set in the support portion/receiving space of the medical device.

All fluid guide systems of the set according to variant a) and/or according to variant b) may be configured to interact with the same medical device after being placed in the support portion/receiving space of the medical device or otherwise fluidically coupled to the medical device.

According to an aspect, a medical device for drug handling may comprise:
A case, and/or
A support portion arranged within the case or on the case and configured to support a removable fluid guide system, preferably a fluid guide system according to any one of the embodiments mentioned above or a fluid guide system of the set as mentioned above, and
At least one electronic control unit (ECU).

The fluid guide system may comprise at least two drug ports configured to be connected to a respective one of at least two drug containers comprising at least one drug. The at least one electronic control unit may be configured to control the operation of the medical device during drug handling using the at least one drug.

The fluid guide system may comprise preferably a fluid channel system. The fluid channel system may comprise at least one channel, at least two channels, at least three channels, etc.

Thus, the medical device (machine) may be configured to reconstitute a drug within the respective drug container and thereafter to pump the reconstituted drug out of the respective drug container, e.g. through the fluid channel system into a secondary or further drug container connected to or connectable to a fluid output port of the fluid guide system, e.g. cartridge or plastic bag.

Alternatively, no reconstitution may be performed but only mixing and/or accumulation of drug(s), e.g. within the further drug container. A combination of reconstitution and/or mixing and/or accumulation of drugs is possible as well.

A pump of the medical device may be configured to provide a fluid flow to transport fluid into an inlet port of a cartridge/flexible carrier (e.g. based on a bag) or interacting with the cartridge/flexible carrier, e.g. with a flexible tube comprising a fluid chamber as mentioned above.

Preferably, a peristaltic pump, a diaphragm pump or another positive replacement pump may be used. However, depending on the kind of drugs, usage of negative pressure pumps may also be considered. The pump may be arranged completely within the device. Alternatively, the pump may be arranged partially within the device and may be mechanically coupled or coupleable to a pump interface of the fluidic guide system, e.g. of a cartridge or of a flexible carrier.

The medical device may perform drug handling automated, e.g. fully automated or partially automated. Thus, drug handling may be failure prove and/or exact, etc. Moreover, costs of HCP (health care personnel) may be reduced considerably, e.g. by enabling a patient to perform the drug handling using the medical device and/or by enabling the patient to perform infusion and/or injection of e.g. the resulting drug mixture or drug liquid himself/herself. The patient may use an infusion port to a vein, artery or another vessel of the human body that has been prepared by HCP. Alternatively, animals, e.g. mammalians may be treated as well.

Thus, the drug or the drugs may be administered, e.g. injected after the drug is or after the drugs are prepared, e.g. using the medical device.

According to an embodiment, the fluid guide system may comprise:
At least one flow control portion configured to control flow in a fluid channel system of the fluid guiding system in a flow-through operation mode and in a blocking operation mode.

The medical device may comprise:
At least one flow control actuator that is configured to interact with the at least one flow control portion of the removable fluid guide system.

The at least one electronic control unit is configured to control the operation of the at least one flow control actuator during drug handling using the at least one drug. The same technical effects as mentioned above for a fluid guide system comprising a flow control portion may apply to the medical device too.

According to an embodiment, the at least one electrical control unit (ECU) may be configured to control the operation of the medical device such that at least two drugs contained in the at least two drug containers coupled to the at least two drug ports are used for the drug handling. The drug handling may include or may consist of reconstitution of at least one of the at least two drugs (D). Alternatively or additionally, other drug handling procedures may be performed, e.g. mixing of drugs and/or accumulation of drugs.

According to an embodiment, the medical device may comprise a positive displacement pump, e.g. a peristaltic pump.

According to a further embodiment, the medical device may comprise:
At least one pump drive unit arranged within the case and configured to drive a pump for providing a fluid flow for transporting fluid through the fluid channel system of the fluid guide system. The at least one electronic control unit may be configured to control the operation of the pump drive unit such that the drug handling/preparation is performed.

Thus, the electronic control unit may perform e.g.:
Switching on and/or switching off the at least one pump, and/or
Changing the pumping direction of the at least one pump, and/or
Controlling the number of revolutions of the pump, e.g. in order to control the resulting fluid flow.

The at least one drug, e.g. a dry drug may be reconstituted within the respective drug container and thereafter be pumped out of the respective drug container, e.g. through the fluid channel system into a secondary or further drug container connected to or connectable to the fluid output port of the fluid guide system, e.g. cartridge or plastic bag. Reconstitution may be performed simultaneously for several or all of the drug containers. Filling of the at least two drug containers may be performed simultaneously or sequentially for some or all of the drug containers. Emptying, e.g. retraction of fluid of the at least two drug containers may be performed simultaneously or sequentially for some or all of the drug containers.

According to an embodiment, the medical device may comprise a movable cover member configured to cover the fluid guide system when the fluid guide system is supported by the device, e.g. a removable fluid guide system. The cover member may comprise at least one or a plurality of openings. Each opening of the plurality of openings may be configured to receive at least one of the drug containers, e.g. a respective one of the drug containers.

Thus, there may be at least 2, 3, 4 or at least 5 openings. The opening may be arranged in a matric, e.g. comprising columns and lines. This may correspond to the matrix that is formed by the drug ports as mentioned above.

The cover member may be configured to pivot about a pivoting axis. Alternatively, the cover member may be configured to slide.

A permanently attached cover member or a detachable cover member may be used. The cover member may form a counteracting surface for at least one valve actuator of the medical device and/or of the fluid guide system. Additionally or alternatively, the cover member may form a counteracting surface for a needle moving mechanism. The cover member may be locked by a locking system/unit during drug handling.

Preferably, the cover, e.g. a pivotable cover may comprise at least one hole or at least two holes configured to allow arrangement of a portion of an inlet tube and the arrangement of a portion of an outlet tube within the at least one hole or at least two holes. The inlet tube may be configured to be fluidically connected or may be fluidically connected to an inlet port of the fluid guide system. The outlet tube may be configured to be fluidically connected or is fluidically connected to an outlet port of the fluid guide system. Two separate holes may allow more freedom in the overall design of the fluid guide system. Alternatively, a common hole may be used, i.e. only one hole. The holes may be through holes, e.g. cylindrical hole(s) and/or having a circular cross section. The portions of the tube may be arranged with play within the hole(s).

This may allow easy removal of the tubes and/or of the fluid guide system from the retaining space. Alternatively, form fit or interference fit may be used to arranged the portion(s) of the flexible tube within the respective hole(s).

The tubes may be flexible tubes. Preferably, the (flexible) inlet tube may be fluidically connected to an auxiliary container, e.g. to a vial or a flexible container (e.g. a plastic bag). Preferably, the (flexible) outlet tube may be fluidically connected to a liquid drug container, e.g. a flexible container (e.g. bag). The technical effects may be the same as mentioned above for the fluid guide system.

According to an embodiment, the medical device may comprise:
At least one movable actuator element configured to move at least one needle arranged within or on the fluid guide system through a closing element of at least one of the at least two drug container. Usage of movable needles may prevent unwanted and/or accidental needle sticks into the body of a user.

According to a further embodiment, alternatively, a stationary needle may be used. Thus, the medical device may not comprise a unit for needle movement, e.g. the medical device may be simpler.

According to a further embodiment, the medical device (machine) may comprise a fluid guide system, e.g. a cartridge or a flexible carrier according to any one of the embodiments mentioned above, preferably a removable and/or consumable fluid guide system.

According to a further embodiment, the electronic control unit of the medical device may be configured to perform at least one of, several of or all of the following steps:
1) Receive input of the user,
2) Generate output for the user,
3) Receive patient data, preferably weight of a patient and/or dose of the drug, e.g. amount of drug necessary for the patient,
4) Select an appropriate program for reconstitution steps and/or for mixing steps and/or drug accumulating steps, e.g. depending on patient data,
5) Calculate a required number of drug containers, e.g. vials, e.g. depending on patient data,
6) Output the number of drug containers needed, e.g. vials and/or output instructions to place the needed number of drug containers, e.g. vials in the respective interface portions or drug ports,
7) Sense the presence of the fluid guide system, e.g. cartridge or carrier comprising the fluid guide system,
8) Actuate at least one lock or latch of the covering member in order to lock the fluid guide system, e.g. cartridge or the carrier comprising the fluid guide system,
9) Sense the presence of the auxiliary container, preferably containing a dilution fluid, e.g. WFI,
10) Sense the presence of the at least one drug container, e.g. of at least one vial,
11) Sense the presence of the at least one secondary or further drug container,
12) Provide feedback of attachment, preferably of the auxiliary container and/or of the at least one drug container and/or of the at least one secondary or further drug container,
13) Guide the user to attach a next item, e.g. the auxiliary container and/or of the at least one drug container and/or of the at least one secondary or further drug container in the correct place,
14) Confirm correct drug containers and/or correct type of drug, e.g. by providing output to a user, preferably of automated detection of the type of drug within the respective drug container, e.g. using RFID (radio frequency identification) tags or prompting the user to confirm that a correct type(s) of drug containers/drug is/are used,
15) Confirm the correct number of drug containers, e.g. by providing output to a user or prompting the user to confirm that a correct number of drug containers are used,
16) Confirm the presence of at least one auxiliary container, preferably of a diluent source, e.g. by providing output to a user or prompting a user to confirm the presence of auxiliary container,
17) Confirm the presence of at least one secondary or further drug container, preferably of an infusion bag, preferably for intravenous (IV) infusion, e.g. by providing output to a user or prompting a user to confirm the presence of the secondary drug container, and/or
18) Perform reconstitution and/or another drug handling, and/or
19) Confirm successful reconstitution or other handling of drugs, e.g. by providing output to a user or prompting the user to confirm that reconstitution/another drug handling was successful, e.g. after visual inspection of the medical device, preferably of all drug containers used.

According to a further embodiment, the medical device may comprise:
A support portion for a drug container, e.g. for a further drug container or for exactly one drug container, preferably for an infusion bag, e.g. for an IV infusion bag. The support portion may be configured to enable support of the drug container in a lying position of the further drug container, preferably of a plastic bag, e.g. filled with a liquid.

Handling of the further drug container may be easier using a flat or curved support surface compared to other supporting options. Moreover, the medical device may be more compact using a horizontal or essentially horizontal support portion. However, hanging of the bag at a holding element, e.g. on a pole may also be a valid option.

Preferably, the support portion may have a size of at least 25 square centimeters, e.g. at least 5cm by at least 5 cm, or of at least 50 square centimeters, e.g. at least 10 cm by at least 5 cm, preferably at most 400 square centimeters. The size may be lower than 700 square centimeters to give only an example for an upper limit. The support portion may comprise a flat support surface or a rounded surface, preferably a concave surface, e.g. in order to provide good support and/or to prevent roll down.

Preferably, the support portion may comprise or may have an area in the range of 80 percent to 150 percent or in the range of 90 percent to 120 percent of the area of a side face of the drug container.

Preferably, the support portion may be movable relative to the case, especially pivotable or slidable. This may allow low storage space if the device is not used or if the device is transported.

The further drug container may be e.g. a plastic bag and/or a plastic box/bottle.

According to a further embodiment, the medical device may comprise:
An auxiliary support portion for at least one an auxiliary fluid container or for exactly one auxiliary fluid container, e.g. comprising a dilution fluid usable for reconstitution of the at least one drug.

The auxiliary support portion may be configured to enable support of the auxiliary drug container in a lying position of the auxiliary drug container, preferably of a plastic bag, e.g. filled with a liquid. The liquid may be a solvent, e.g. WFI (water for injection).

The auxiliary support portion may comprise a flat surface or a rounded surface, e.g. a concave surface. The auxiliary container may be a plastic bag. Handling of the auxiliary container may be easier using a flat or curved support surface compared to other supporting options. However, hanging of the auxiliary bag at a holding element, e.g. on a pole may also be a valid option.

Alternatively, the at least one auxiliary container may comprise at least one vial or another rigid drug container. The device may be adaptable to different types of auxiliary container(s).

According to a further embodiment, the medical device may comprise at least one display unit, preferably a pivotable display unit. The display unit may be configured to communicate output to a user as is mentioned below in more detail. The display unit may comprise a screen, e.g. comprising at least 200 pixels (picture elements) or at least 1000 pixels. Thus, the display unit may be used to instruct the user using written words, e.g. for acoustically impaired users. This may enable communication of longer instructions. Alternatively or additionally, acoustic means may be used to communicate information from the device to the user, e.g. for visibly impaired users.

The display unit may comprise a touchscreen, e.g. enabling the user to make inputs. However, an LED (light emission diode) display or an LCD (liquid crystal display) may be used as well.

According to an embodiment, a method for drug handling is provided, comprising:
Preferably, using a fluid guide system that comprises a fluid channel system and at least two drug ports, each drug port being configured to receive a fluid comprising at least one drug from at least one drug container, and/or
Preferably, using a medical device comprising a supporting portion for the fluid guide system,
Preferably, mechanically coupling the at least two drug containers to the at least two drug ports jointly during handling of the at least drug, and/or
Preferably, applying the fluid guide system to the supporting portion of the device before or after coupling, and/or
Performing at least on drug handling step automatically using the at least one medical device when the fluid guide system is applied to the supporting portion of the device.

Preferably, the medical device may be a medical device as mentioned in any one of the embodiments above.

According to an embodiment, the fluid guide system may be the fluid guide system according to any one of the embodiments mentioned above or a fluid guide system of a set according to the embodiment mentioned above.

According to an aspect, a computer-implemented method is provided comprising:
Sensing or prompting a user to confirm the coupling of at least two drug containers to a respective one of at least two drug ports of a fluid guide system.

The least two drug ports may be configured to receive a fluid comprising at least one drug from at least one respective drug container. Preferably, the computer-implemented method may be performed by (the) at least one electronic control unit of the medical device according to any one of the embodiments as mentioned above.

According to an embodiment, the computer-implemented method may comprise at least one of, several of or all of:
Controlling a pump drive unit that is configured to drive a pump, wherein at least a part of the pump is implemented on the fluid guide system or wherein the pump is configured to generate the pumping force for a fluid flow within the fluid guide system, and/or
Prompting a user to begin a drug handling process, preferably a reconstitution and/or a mixing process, and/or
Performing a calculation to calculate a dose of the drug, preferably depending on parameters of the user, e.g. weight, state of a disease, etc., and/or
Providing alerts and/or guidance to the user, preferably to perform a correct sequence of steps and/or to avoid errors (e.g. to avoid hazards), and/or
Sensing the presence of at least one drug container, preferably of at least one of:
   An auxiliary drug container, preferably comprising a diluent, and/or
   A drug container comprising the at least one drug, preferably in a lyophilized state, and/or
   A further drug container configured to receive the at least one drug, preferably in a reconstituted state, e.g. a flexible bag, preferably an infusion bag.

The computer implemented method may perform at least one, several or all of the steps 1) to 19) as mentioned above, e.g.: 1) Receive input from the user, 2) Generate output for the user, etc.

According to a further aspect, a data processing device is provided, preferably a computer comprising a processor for carrying out any one of the computer implemented methods mentioned above. Thus, the same technical effects as mentioned above may apply.

The computer, e.g. the ECU as mentioned above may comprise:
The processor, and/or
At least one memory, e.g. a non-volatile storing memory and/or a volatile storing memory, and/or
At least one input unit, and/or
At least one output unit, and/or
At least one electronic interface to an actuator element, e.g. to a valve actuator and/or to a pump.

According to a further aspect, a computer program product is provided comprising instructions which, when the program is executed by a computer, cause the computer to carry out any one of the computer implemented methods mentioned above. Thus, the same technical effects apply.

The instructions may be instructions which may be performed directly by a processor, e.g. machine instructions, preferably binary coded instructions. Alternatively, the instructions may be instructions of a higher computer language e.g. Assembly language, C, C#, Python, etc.

According to a further aspect, a computer readable medium is provided comprising instructions which, when executed by a computer, cause the computer to carry out any one of the computer implemented methods mentioned above.

According to a further aspect, administering of a drug or of a drug mixture is provided that was prepared using the systems/methods mentioned above, e.g. the fluid guide system, the medical device, etc. Thus, the drug or the drugs may be administered, e.g. injected after the drug is or after the drugs are prepared, e.g. using the fluid guide system, the medical device or the method according to any one of the embodiments mentioned above.

The instructions may be the same instructions as mentioned above for the computer program product. The computer readable medium may be e.g. a storage medium for storage of digital data.

According to a further aspect a data transmission sequence is provided comprising a sequence of electrical signals or electromagnetic waves for the transmission of the computer program product via conductive or non-conductive line, e.g. optical transmission line (wired) or wireless.

In the following, some general remarks are mentioned before details of embodiments of the invention are described below. The general remarks refer to:
1. List of drugs
2. Definitions
3. First set of aspects (general)
4. Second set of aspects (more specific)
5. Features which may be applied to any embodiment

### 1. List of Drugs

The terms “drug” or “medicament” are used synonymously herein and describe a pharmaceutical formulation containing one or more active pharmaceutical ingredients or pharmaceutically acceptable salts or solvates thereof, and optionally a pharmaceutically acceptable carrier. An active pharmaceutical ingredient (“API”), in the broadest terms, is a chemical structure that has a biological effect on humans or animals. In pharmacology, a drug or medicament is used in the treatment, cure, prevention, or diagnosis of disease or used to otherwise enhance physical or mental well-being. A drug or medicament may be used for a limited duration, or on a regular basis for chronic disorders. As described below, a drug or medicament can include at least one API, or combinations thereof, in various types of formulations, for the treatment of one or more diseases. Examples of API may include small molecules having a molecular weight of 500 Da or less; polypeptides, peptides and proteins (e.g., hormones, growth factors, antibodies, antibody fragments, and enzymes); carbohydrates and polysaccharides; and nucleic acids, double or single stranded DNA (including naked and cDNA), RNA, antisense nucleic acids such as antisense DNA and RNA, small interfering RNA (siRNA), ribozymes, genes, and oligonucleotides. Nucleic acids may be incorporated into molecular delivery systems such as vectors, plasmids, or liposomes. Mixtures of one or more drugs are also contemplated.

The drug or medicament may be contained in a primary package or “drug container” adapted for use with a drug delivery device. The drug container may be, e.g., a cartridge, syringe, reservoir, or other solid or flexible vessel configured to provide a suitable chamber for storage (e.g., short- or long-term storage) of one or more drugs. For example, in some instances, the chamber may be designed to store a drug for at least one day (e.g., 1 to at least 30 days). In some instances, the chamber may be designed to store a drug for about 1 month to about 2 years. Storage may occur at room temperature (e.g., about 20° C.), or refrigerated temperatures (e.g., from about −4° C. to about 4° C.). In some instances, the drug container may be or may include a dual −chamber cartridge configured to store two or more components of the pharmaceutical formulation to-be-administered (e.g., an API and a diluent, or two different drugs) separately, one in each chamber. In such instances, the two chambers of the dual-chamber cartridge may be configured to allow mixing between the two or more components prior to and/or during dispensing into the human or animal body. For example, the two chambers may be configured such that they are in fluid communication with each other (e.g., by way of a conduit between the two chambers) and allow mixing of the two components when desired by a user prior to dispensing. Alternatively or in addition, the two chambers may be configured to allow mixing as the components are being dispensed into the human or animal body.

The drugs or medicaments contained in the drug delivery devices as described herein can be used for the treatment and/or prophylaxis of many different types of medical disorders. Examples of disorders include, e.g., diabetes mellitus or complications associated with diabetes mellitus such as diabetic retinopathy, thromboembolism disorders such as deep vein or pulmonary thromboembolism. Further examples of disorders are acute coronary syndrome (ACS), angina, myocardial infarction, cancer, macular degeneration, inflammation, hay fever, atherosclerosis and/or rheumatoid arthritis. Examples of APIs and drugs are those as described in handbooks such as Rote Liste 2014, for example, without limitation, main groups 12 (anti-diabetic drugs) or 86 (oncology drugs), and Merck Index, 15th edition.

Examples of APIs for the treatment and/or prophylaxis of type 1 or type 2 diabetes mellitus or complications associated with type 1 or type 2 diabetes mellitus include an insulin, e.g., human insulin, or a human insulin analogue or derivative, a glucagon-like peptide (GLP-1), GLP-1 analogues or GLP-1 receptor agonists, or an analogue or derivative thereof, a dipeptidyl peptidase-4 (DPP4) inhibitor, or a pharmaceutically acceptable salt or solvate thereof, or any mixture thereof. As used herein, the terms “analogue” and “derivative” refers to a polypeptide which has a molecular structure which formally can be derived from the structure of a naturally occurring peptide, for example that of human insulin, by deleting and/or exchanging at least one amino acid residue occurring in the naturally occurring peptide and/or by adding at least one amino acid residue. The added and/or exchanged amino acid residue can either be codable amino acid residues or other naturally occurring residues or purely synthetic amino acid residues. Insulin analogues are also referred to as “insulin receptor ligands”. In particular, the term “derivative” refers to a polypeptide which has a molecular structure which formally can be derived from the structure of a naturally occurring peptide, for example that of human insulin, in which one or more organic substituent (e.g. a fatty acid) is bound to one or more of the amino acids. Optionally, one or more amino acids occurring in the naturally occurring peptide may have been deleted and/or replaced by other amino acids, including non-codeable amino acids, or amino acids, including non-codeable, have been added to the naturally occurring peptide.

Examples of insulin analogues are Gly(A21), Arg(B31), Arg(B32) human insulin (insulin glargine); Lys(B3), Glu(B29) human insulin (insulin glulisine); Lys(B28), Pro(B29) human insulin (insulin lispro); Asp(B28) human insulin (insulin aspart); human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Examples of insulin derivatives are, for example, B29-N-myristoyl-des(B30) human insulin, Lys(B29) (N-tetradecanoyl)-des(B30) human insulin (insulin detemir, Levemir®); B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N-(N-palmitoyl-gamma-glutamyl)-des(B30) human insulin, B29-N-omega-carboxypentadecanoyl-gamma-L-glutamyl-des(B30) human insulin (insulin degludec, Tresiba®); B29-N-(N-lithocholyl-gamma-glutamyl)-des(B30) human insulin; B29-N-(ω-carboxyheptadecanoyl)-des(B30) human insulin and B29-N-(ω-carboxyheptadecanoyl) human insulin.

Examples of GLP-1, GLP-1 analogues and GLP-1 receptor agonists are, for example, Lixisenatide (Lyxumia®), Exenatide (Exendin-4, Byetta®, Bydureon®, a 39 amino acid peptide which is produced by the salivary glands of the Gila monster), Liraglutide (Victoza®), Semaglutide, Taspoglutide, Albiglutide (Syncria®), Dulaglutide (Trulicity®), rExendin-4, CJC-1134-PC, PB-1023, TTP-054, Langlenatide/HM-11260C (Efpeglenatide), HM-15211, CM-3, GLP-1 Eligen, ORMD-0901, NN-9423, NN-9709, NN-9924, NN-9926, NN-9927, Nodexen, Viador-GLP-1, CVX-096, ZYOG-1, ZYD-1, GSK-2374697, DA-3091 March-701, MAR709, ZP-2929, ZP-3022, ZP-DI-70, TT-401 (Pegapamodtide), BHM-034. MOD-6030, CAM-2036, DA-15864, ARI-2651, ARI-2255, Tirzepatide (LY3298176), Bamadutide (SAR425899), Exenatide-XTEN and Glucagon-Xten.

An example of an oligonucleotide is, for example: mipomersen sodium (Kynamro®), a cholesterol-reducing antisense therapeutic for the treatment of familial hypercholesterolemia or RG012 for the treatment of Alport syndrom.

Examples of DPP4 inhibitors are Linagliptin, Vildagliptin, Sitagliptin, Denagliptin, Saxagliptin, Berberine.

Examples of hormones include hypophysis hormones or hypothalamus hormones or regulatory active peptides and their antagonists, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, and Goserelin.

Examples of polysaccharides include a glucosaminoglycane, a hyaluronic acid, a heparin, a low molecular weight heparin or an ultra-low molecular weight heparin or a derivative thereof, or a sulphated polysaccharide, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium. An example of a hyaluronic acid derivative is Hylan G-F 20 (Synvisc®), a sodium hyaluronate.

The term “antibody”, as used herein, refers to an immunoglobulin molecule or an antigen-binding portion thereof. Examples of antigen-binding portions of immunoglobulin molecules include F(ab) and F(ab′)2 fragments, which retain the ability to bind antigen. The antibody can be polyclonal, monoclonal, recombinant, chimeric, de-immunized or humanized, fully human, non-human, (e.g., murine), or single chain antibody. In some embodiments, the antibody has effector function and can fix complement. In some embodiments, the antibody has reduced or no ability to bind an Fc receptor. For example, the antibody can be an isotype or subtype, an antibody fragment or mutant, which does not support binding to an Fc receptor, e.g., it has a mutagenized or deleted Fc receptor binding region. The term antibody also includes an antigen-binding molecule based on tetravalent bispecific tandem immunoglobulins (TBTI) and/or a dual variable region antibody-like binding protein having cross-over binding region orientation (CODV).

The terms “fragment” or “antibody fragment” refer to a polypeptide derived from an antibody polypeptide molecule (e.g., an antibody heavy and/or light chain polypeptide) that does not comprise a full-length antibody polypeptide, but that still comprises at least a portion of a full-length antibody polypeptide that is capable of binding to an antigen. Antibody fragments can comprise a cleaved portion of a full length antibody polypeptide, although the term is not limited to such cleaved fragments. Antibody fragments that are useful in the present invention include, for example, Fab fragments, F(ab′)2 fragments, scFv (single-chain Fv) fragments, linear antibodies, monospecific or multispecific antibody fragments such as bispecific, trispecific, tetraspecific and multispecific antibodies (e.g., diabodies, triabodies, tetrabodies), monovalent or multivalent antibody fragments such as bivalent, trivalent, tetravalent and multivalent antibodies, minibodies, chelating recombinant antibodies, tribodies or bibodies, intrabodies, small modular immunopharmaceuticals (SMIP), binding-domain immunoglobulin fusion proteins, camelized antibodies, and immunoglobulin single variable domains. Additional examples of antigen-binding antibody fragments are known in the art.

The term “immunoglobulin single variable domain” (ISV), interchangeably used with “single variable domain”, defines immunoglobulin molecules wherein the antigen binding site is present on, and formed by, a single immunoglobulin domain. As such, immunoglobulin single variable domains are capable of specifically binding to an epitope of the antigen without pairing with an additional immunoglobulin variable domain. The binding site of an immunoglobulin single variable domain is formed by a single heavy chain variable domain (VH domain or VHH domain) or a single light chain variable domain (VL domain). Hence, the antigen binding site of an immunoglobulin single variable domain is formed by no more than three CDRs.

An immunoglobulin single variable domain (ISV) can be a heavy chain ISV, such as a VH (derived from a conventional four-chain antibody), or VHH (derived from a heavy-chain antibody), including a camelized VH or humanized VHH. For example, the immunoglobulin single variable domain may be a (single) domain antibody, a “dAb” or dAb or a Nanobody® ISV (such as a VHH, including a humanized VHH or camelized VH) or a suitable fragment thereof. [Note: Nanobody® is a registered trademark of Ablynx N.V.]; other single variable domains, or any suitable fragment of any one thereof.

“VHH domains”, also known as VHHs, VHH antibody fragments, and VHH antibodies, have originally been described as the antigen binding immunoglobulin variable domain of “heavy chain antibodies” (i.e., of “antibodies devoid of light chains”; Hamers-Casterman et al. 1993 (Nature 363: 446-448). The term “VHH domain” has been chosen in order to distinguish these variable domains from the heavy chain variable domains that are present in conventional 4-chain antibodies (which are referred to herein as “VH domains”) and from the light chain variable domains that are present in conventional 4-chain antibodies (which are referred to herein as “VL domains”). For a further description of VHH's, reference is made to the review article by Muyldermans 2001 (Reviews in Molecular Biotechnology 74: 277-302).

For the term “dAb's” and “domain antibody”, reference is for example made to Ward et al. 1989 (Nature 341: 544), to Holt et al. 2003 (Trends Biotechnol. 21: 484); as well as to WO 2004/068820, WO 2006/030220, WO 2006/003388. It should also be noted that, although less preferred in the context of the present invention because they are not of mammalian origin, single variable domains can be derived from certain species of shark (for example, the so-called “IgNAR domains”, see for example WO 2005/18629).

The terms “Complementarity-determining region” or “CDR” refer to short polypeptide sequences within the variable region of both heavy and light chain polypeptides that are primarily responsible for mediating specific antigen recognition. The term “framework region” refers to amino acid sequences within the variable region of both heavy and light chain polypeptides that are not CDR sequences, and are primarily responsible for maintaining correct positioning of the CDR sequences to permit antigen binding. Although the framework regions themselves typically do not directly participate in antigen binding, as is known in the art, certain residues within the framework regions of certain antibodies can directly participate in antigen binding or can affect the ability of one or more amino acids in CDRs to interact with antigen.

Examples of antibodies are anti PCSK-9 mAb (e.g., Alirocumab), anti IL-6 mAb (e.g., Sarilumab), and anti IL-4 mAb (e.g., Dupilumab).

Pharmaceutically acceptable salts of any API described herein are also contemplated for use in a drug or medicament in a drug delivery device. Pharmaceutically acceptable salts are for example acid addition salts and basic salts.

Those of skill in the art will understand that modifications (additions and/or removals) of various components of the APIs, formulations, apparatuses, methods, systems and embodiments described herein may be made without departing from the full scope and spirit of the present invention, which encompass such modifications and any and all equivalents thereof.

An example drug delivery device may involve a needle-based injection system as described in Table 1 of section 5.2 of ISO 11608-1:2014(E). As described in ISO 11608-1:2014(E), needle-based injection systems may be broadly distinguished into multi-dose container systems and single-dose (with partial or full evacuation) container systems. The container may be a replaceable container or an integrated non-replaceable container.

As further described in ISO 11608-1:2014(E), a multi-dose container system may involve a needle-based injection device with a replaceable container. In such a system, each container holds multiple doses, the size of which may be fixed or variable (pre-set by the user). Another multi-dose container system may involve a needle-based injection device with an integrated non-replaceable container. In such a system, each container holds multiple doses, the size of which may be fixed or variable (pre-set by the user).

As further described in ISO 11608-1:2014(E), a single-dose container system may involve a needle-based injection device with a replaceable container. In one example for such a system, each container holds a single dose, whereby the entire deliverable volume is expelled (full evacuation). In a further example, each container holds a single dose, whereby a portion of the deliverable volume is expelled (partial evacuation). As also described in ISO 11608-1:2014(E), a single-dose container system may involve a needle-based injection device with an integrated non-replaceable container. In one example for such a system, each container holds a single dose, whereby the entire deliverable volume is expelled (full evacuation). In a further example, each container holds a single dose, whereby a portion of the deliverable volume is expelled (partial evacuation).

### 2. Definitions

A “vial” (also known as a phial or flacon) may be a small glass or plastic vessel or bottle, often used to store medication in the form of liquids, powders, or capsules. Modern vials may be made of plastics such as polypropylene (PP). Vials may comprise a volume in the range of 1 ml (milliliter) to 100 ml or in the range of 5 ml to 40 ml. Vials may be closed by a rubber or plastic plug or by a septum (e.g. membrane that can be pierced through by a needle), e.g. a PTFE (polytetrafluoroethylene) septum. The vial may comprise a smaller neck portion near its opening. A crimped metal cap, e.g. aluminum may be used to secure the plug. The crimped cap may be secured at the neck portion.

A “flexible carrier” may be a flexible container made of a thin sheet or foil, e.g. plastic. The thickness may be in the range of 25 micrometer to 100 micrometer. Alternatively, the thickness may be more than 100 micrometer. Heat sealing may be used to produce the plastic carrier.

An “infusion bag” may be a flexible carrier, e.g. a plastic carrier that is used for infusion. The infusion bag may have at least one port, e.g. a Luer Lock port, a self-sealing infusion port, medication port, spiking port, IV tubing port, etc. The infusion bag may have at least two ports. An integrated hanger opening may be located at an edge of the infusion bag. Thus, the term “infusion bag” or “infusion pouch” may refer to a plastic carrier used for infusion. Gravity may be used to empty the infusion bag slowly. A clamp may be used to regulate the fluid flow out of the infusing carrier. An alternative may be an electromagnetic infusion pump, a latex balloon or other higher pressure container, e.g. higher than the atmospheric pressure, a mechanical “platen” pump, etc. All of these containers may be used in the described embodiments.

“Intravenous therapy” (abbreviated as IV therapy) may be a medical technique that administers fluids, medications and/or nutrients directly into a person's vein. The intravenous route of administration may also be used to administer medications or other medical therapy such as blood products or electrolytes to correct electrolyte imbalances. There are several IV containers, e.g. plastic carrier, plastic bottle, etc. All of these containers may be used in the described embodiments.

“Intra-arterial” infusion may be used as well, e.g. under certain circumstances. However, intravenous infusion may be preferred for medical reasons, e.g. lower blood pressure in veins compared to blood pressure in arteries. Thus, all embodiments may also refer to intra-arterial infusion.

“Lyophilization”, also known as freeze drying, may be used for preserving biological material by removing the water from the sample, which may involve first freezing the sample and then drying it, under vacuum, at very low temperatures, e.g. at a temperature in the range of −30 C (degrees Celsius, centigrade) to −50° C. or in the range of −35 to −45° C., e.g. at −40° C. or at around/about −40° C. The lyophilization process may take 10 to 75 hours to go through all of these stages.

“Lyophilized products” in biotechnology and pharmaceuticals may include injectable solutions, proteins, oligonucleotides, peptides, and vaccines.

“Reconstitution” of lyophilized drug products may be performed before it can be administered. In simple terms this involves mixing with a fixed amount of water-based liquid (i.e., diluent), preferably sterile water and waiting and/or agitating the resulting mixture until the drug product is dissolved or dispersed.

The term “disease”, as used herein, refers to an abnormal condition that affects the body of an individual. A disease is often construed as a medical condition associated with specific symptoms and signs. In humans, the term “disease” is often used more broadly to refer to any condition that causes pain, dysfunction, distress, social problems, or death to the individual afflicted, or similar problems for those in contact with the individual. In this broader sense, it sometimes includes injuries, disabilities, disorders, syndromes, infections, deviant behaviors, and atypical variations of structure and function, while in other contexts and for other purposes these may be considered distinguishable categories. Diseases usually affect individuals not only physically, but also emotionally, as contracting and living with many diseases can alter one's perspective on life, and one's personality. Examples of diseases are “autoimmune disease”, “cardiovascular disease”, “fibrotic disease”, “infectious disease”, “respiratory disease”, “inflammatory disease”, “cancer disease” or “cancer”, “diabetes”, etc.

The term “diabetes” or “diabetes mellitus”, as used herein, refers to or describes a group of common endocrine diseases characterized by sustained high blood sugar levels. Diabetes may be due to either the pancreas not producing enough insulin, or the cells of the body becoming unresponsive to the hormone's effects. There are several types of diabetes, e.g. type 1 and type 2.

The at least one drug or medicament may be used to heal or to mitigate any disease, e.g. any disease mentioned in this application.

The term “drug handling” refers to the reconstitution and/or to the mixing of drugs, e.g. in order to prepare them for administration. Drug handling may be done e.g. immediately before administering (e.g. injecting) the drug after the drug is prepared. Thus, the time between the end of drug handling and the beginning of administration, e.g. injection, inhalation, etc. may be less than 5 hours, less than 2 hours, less than 1 hour, less than 30 minutes or less than 10 minutes.

### 3. First Set of Aspects (General)

In the following, a first set of aspects is disclosed. The aspects are numbered to facilitate referencing the features of one aspect in other aspects. The aspects form part of the disclosure of the present application and could be made subject to independent and/or dependent claims irrespective of what is claimed in the application currently.

Aspect 1. A fluid guide system usable for drug handling, comprising:
at least one fluid port configured to dispense a fluid from the fluid guide system,
at least one drug port configured to receive a fluid comprising at least one drug from at least one drug container.

A carrier of the fluid guide system, especially of a fluid channel system of the fluid guide system may be rigid or flexible. The carrier may be configured to carry the at least one fluid port and the at least one drug port, preferably also a fluid channel system connecting the at least one fluid port and the at least one drug port fluidically, e.g. in at least one operation mode or operation state of the fluid guide system.

Aspect 2. The fluid guide system according to aspect 1, comprising at least one flow control portion,
wherein the at least one drug port is fluidically connected or connectable to the at least one fluid port via the at least one flow control portion, and
wherein the at least one flow control portion is configured to allow a fluid flow through the at least one flow control portion in a flow-through operation mode of the at least one flow control portion and to prevent a fluid flow through the at least flow control portion in a blocking operation mode of the flow control portion.

The flow control portion may be switchable between the flow-through operation mode and the blocking operation mode.

Aspect 3. The fluid guide system according to aspect 1 or 2, wherein the at least one drug port comprises at least two drug ports,
wherein each of the at least two drug ports is coupleable or coupled to at least one drug container such that a plurality of drug containers can be coupled simultaneously to the fluid guide system.

The coupling may be preferably a mechanical coupling. However, filling or emptying of drug containers may occur sequentially or simultaneously.

Aspect 4. The fluid guide system according to any one of aspects 1 to 3, wherein the fluid guide system is configured to transport drug from a plurality of the drug containers via a (e.g. common) channel system of the fluid guide system into at least one (e.g. exactly one) common drug container.

Aspect 5. The fluid guide system according to any one of aspects 1 to 4, comprising at least one of:
an inlet port configured to be fluidically connected to an auxiliary container, preferably to an auxiliary container comprising a diluent, e.g. WFI (water for injection), and/or
an outlet port configured to be fluidically connected to a further drug container, preferably to a further drug container comprising a saline solution and/or configured to contain the handled drug(s), and/or
a common input/output port configured to be fluidically connected to the auxiliary container and to the further drug container.

Aspect 6. The fluid guide system according to aspect 4, wherein the fluid guide system is configured such that at least two drug containers of the plurality of drug containers are coupled sequentially to a common drug port of the fluid guide system.

Aspect 7. The fluid guide system according to aspect 6, wherein the fluid guide system is configured such that the at least two drug containers are moved relative to the common drug port.

A hopper, a transport unit, a conveyer, etc. may be used for moving of the drug containers. Thus, e.g. a vial moving element may be used. The vial moving element may be an increment, e.g. wheel with acute teeth.

Aspect 8. The fluid guide system according to aspect 6, wherein the fluid guide system is configured such that the common drug port is moved relative to the at least two drug containers.

A moving head may be movable in one dimension (1D, linear), in two dimensions (2D) or in three dimensions (3D). The drug containers may be arranged stationary relative to fluid guide system and/or to a case of a rigid fluid guide system, e.g. a cartridge. Alternatively, the drug containers may be arranged stationary to the position of a flexible carrier comprising the fluid guide system.

Aspect 9. The fluid guide system according to any one of aspects 1 to 8, wherein the fluid guide system is a fluid guide system according to any one of the appended claims or wherein the fluid guide system is configured to interact with the medical device according to one of the appended claims.

Aspect 10. A medical device for drug handling, e.g. for drug reconstitution, comprising:
a case, and/or
a support portion/receiving space arranged within the case or on the case and configured to support/receive a removable fluid guide system, and/or
wherein the fluid guide system comprises preferably a fluid channel system and at least one drug port configured to be connected to a respective one of at least one drug container comprising at least one drug, and/or
at least one electronic control unit that is configured to control the operation of the medical device during drug handling using the at least one drug.

Aspect 11. The medical device, according to aspect 10,
wherein the fluid guide system comprises at least one flow control portion configured to control flow in a fluid cannel system in a flow-through operation mode and in a blocking operation mode, and wherein
the medical device comprises:
at least one flow control actuator that is configured to interact with the at least one flow control portion of the removable fluid guide system, and
wherein the at least one electrical control unit is configured to control the operation of the at least one flow control actuator during drug preparation using the at least one drug.

Aspect 12. The medical device according to aspect 10 or 11, wherein the fluid guide system comprises at least two drug ports, and/or
wherein the at least one electrical control unit is configured to control the operation of the medical device such that at least two drugs contained in at least two drug containers coupled to the at least two drug ports are used for the drug handling/preparation, and/or
preferably including reconstitution of at least one of the at least two drugs and/or mixing/accumulating of the at least two drugs.

Aspect 13. The medical device according to any one of aspects 10 to 12, wherein the fluid guide system is a fluid guide system according to any one of the aspects 1 to 9.

Aspect 14. A method for drug handling, comprising:
using a fluid guide system that comprises preferably a fluid channel system and at least one drug portion configured to receive a fluid comprising at least one drug from at least one drug container, and/or
using a medical device comprising a support portion/receiving space for the fluid guide system and an electronic control unit, and/or
controlling the operation of the medical device by the electronic control unit.

Aspect 15. The method according to aspect 14, wherein the fluid guide system comprises at least one flow control portion,
wherein the medical device comprises at least one flow control actuator configured to interact with the at least one flow control portion of the fluid guide system, and/or
wherein controlling comprises controlling the operation of the medical device by the electronic control unit such that the at least one drug is reconstituted and/or used for the drug handling thereby switching an operation mode of the at least one control portion, e.g. from a blocking operation mode to a flow-through operation mode or from the flow-through operation mode to the blocking operation mode at least once using the at least one flow control actuator.

Aspect 16. The method according to aspect 14 or 15, wherein the at least one drug port comprises at least two drug ports, and
wherein each of the at least two drug ports is coupleable or coupled to at least one drug container such that a plurality of drug containers can be coupled simultaneously to the fluid guide system.

Aspect 17. The method according to any one of aspects 14 to 16, wherein the fluid guide system is a fluid guide system according to any one of the aspects 1 to 9 or wherein the medical device is a medical device according to any one of the aspects 10 to 13.

Features mentioned in the description, e.g. in the second set of aspects or feature groups (FG) or in the appended claims for the fluid guide system, set, medical device, method, etc. may be applied to any one of the first set of aspects and vice versa.

### 4. Second Set of Aspects (More Specific)

In the following, a second set of aspects is disclosed. The aspects are numbered to facilitate referencing the features of one aspect in other aspects. The aspects form part of the disclosure of the present application and could be made subject to independent and/or dependent claims irrespective of what is claimed in the application currently.

Aspect 1. A fluid guide system usable for drug handling, comprising:
at least one fluid port configured to dispense a fluid from the fluid guide system,
at least two drug ports, each drug port being configured to receive a fluid comprising at least one drug from at least one drug container,
wherein each of the at least two drug ports is coupleable or coupled to at least one drug container such that a plurality of drug containers can be or is jointly mechanically coupled to the fluid guide system.

Aspect 2. The fluid guide system according to aspect 1, comprising:
a fluid channel system, and
a carrier configured to carry the fluid channel system,
wherein the at least two drug ports comprise a respective fluid transmitting portion configured to transmit fluid coming from the respective drug port of the at least two drug ports,
wherein the fluid channel system comprises the at least one fluid port and the fluid portions of the at least two drug ports, and
wherein the fluid channel system is configured to be supported by a medical device.

Aspect 3. The fluid guide system according to aspect 2, wherein the carrier comprises a rigid cartridge,
wherein the rigid cartridge comprises a rigid case, and
wherein the rigid case comprises at least two openings configured to allow access to the at least two drug ports in order to couple a respective one of the plurality of drug containers to a respective one of the at least two drug ports.

Aspect 4. The fluid guide system according to aspect 2, wherein the carrier comprises a flexible carrier portion,
wherein at least a part of the fluid channel system is formed integrally with the flexible carrier by connecting at least two flexible sheet materials selectively thereby forming the fluid channel system.

Aspect 5. The fluid guide system according to any one of the preceding aspects, comprising at least one flow control portion,
wherein at least one of the at least two drug port is or wherein the at least two drug ports are fluidically connected or connectable to the at least one fluid port via the at least one flow control portion, and
wherein the at least one flow control portion is configured to allow a fluid flow through the at least one flow control portion in a flow-through operation mode of the at least one flow control portion and to prevent a fluid flow through the at least flow control portion in a blocking operation mode of the flow control portion.

The fluid guide system, according to any one of the preceding aspects, wherein the at least one drug portion comprise at least one respective needle,
wherein the respective needle is arranged movably relative to a case of the fluid guide system or wherein the respective needle may be arranged within or on a flexible bag that forms the carrier of the fluid guide system.

According to a further aspect, the fluid guide system according to aspect 5 may comprise at least two flow control portions of the at least one flow control portion,
wherein a fluid flow to each one of the at least two drug ports is controllable via the operation mode of a respective one of the at least two flow control portions.

Aspect 7. The fluid guide system according to any one of the preceding aspects, wherein the at least two drug ports comprise at least four drug ports,
wherein the at least for drug ports are arranged in a matrix comprising at least two columns and at least two lines.

Aspect 8. The fluid guide system according to any one of the preceding aspects, comprising at least one channel extending along a plurality of the at least two drug ports, and
at least two auxiliary channels connecting a respective drug port of the at least two drug ports fluidically with the channel,
wherein the respective auxiliary channel and the channel form an angle within the range of 10 degrees to 80 degrees, in the range of 20 degrees to 70 degrees or in the range of 30 degrees to 60 degrees.

Aspect 9. The fluid guide system according to any one of the preceding aspects comprising
at least one output port configured to be connected or connectable to a further drug container, and
at least one input port configured to be connected or connectable to an auxiliary drug container,
wherein the at least output port and at least one input port are both fluidically connectable or connected to at least one intermediate fluid channel of the fluid guide system,
wherein a) the fluid guide system comprises a pump interface unit selectively coupleable to the at least one input port and to the at least one output port, or
wherein b) the fluid guide system comprises a pump selectively coupleable to the at least one input port and to the at least one output port.

Aspect 10. A set of fluid guide systems, comprising at least one fluid guide system according to any one of the preceding aspects, and at least one of or both the following:
a) at least one fluid guide module which is configured to be coupled to at least one of the at least two drug ports via a common coupling port,
wherein the at least one fluid guide module comprises at least two further drug ports,
wherein the at least two further drug ports are fluidically coupled or coupleable to the common coupling port of the at least one modular fluid guide system, and/or
b) at least one further fluid guide system, wherein the further fluid guide system is configured to be applied to the same medical device as the fluid guide system according to any one of the preceding aspects, and
wherein the further fluid guide system comprises compared to the fluid guide system according to any one of the preceding aspects at least one, several of or all of:
a different fluid channel system,
a different number of fluid control portions,
a different number of drug ports,
a different size,
a different outer shape.

Aspect 11. A medical device for drug handling, comprising:
a case,
a support portion arranged within the case or on the case and configured to support a removable fluid guide system, preferably a fluid guide system according to any one of aspects 1 to 9 or a fluid guide system of the set according to aspect 10, and
at least one electronic control unit,
wherein the fluid guide system comprises at least two drug ports configured to be connected to a respective one of at least two drug containers comprising at least one drug, and
wherein the at least one electronic control unit is configured to control the operation of the medical device during drug handling using the at least one drug.

Aspect 12. The medical device according to aspect 11,
wherein the fluid guide system comprises at least one flow control portion configured to control flow in a fluid channel system of the fluid guiding system in a flow-through operation mode and in a blocking operation mode, and wherein
the medical device comprises:
at least one flow control actuator that is configured to interact with the at least one flow control portion of the removable fluid guide system, and
wherein the at least one electronic control unit is configured to control the operation of the at least one flow control actuator during drug handling using the at least one drug.

Aspect 13. The medical device according to aspect 11 or 12,
wherein the at least one electrical control unit is configured to control the operation of the medical device such that at least two drugs contained in the at least two drug containers coupled to the at least two drug ports are used for the drug handling,
wherein the drug handling includes reconstitution of at least one of the at least two drugs (D).

Aspect 14. The medical device according to any one of aspects 11 to 13, comprising a positive displacement pump, e.g. a peristaltic pump.

Aspect 15. The medical device according to any one of aspects 11 to 14, comprising a movable cover member configured to cover the fluid guide system when the fluid guide system is supported by the device,
wherein the cover member comprises a plurality of openings, and
wherein each opening of the plurality of openings is configured to receive a drug container.

Aspect 16. The medical device according to any one of aspects 10 to 14, comprising:
at least one movable actuator element configured to move at least one needle arranged within or on the fluid guide system through a closing element of at least one of the at least two drug container.

Aspect 17. A method for drug handling, comprising:
using a fluid guide system that comprises a fluid channel system and at least two drug ports, each drug port being configured to receive a fluid comprising at least one drug from at least one drug container,
using a medical device comprising a supporting portion for the fluid guide system,
mechanically coupling the at least two drug containers to the at least two drug ports jointly during handling of the at least drug,
applying the fluid guide system to the supporting portion of the device before or after coupling,
performing at least on drug handling step automatically using the at least one medical device when the fluid guide system is applied to the supporting portion of the device.

Aspect 18. The method according to aspect 17, wherein the fluid guide system is the fluid guide system according to any one of the aspects 1 to 9 or a fluid guide system of a set according to aspect 10 and/or wherein the medical device is the medical device according to any one of the aspects 11 to 16.

Aspect 19. A computer-implemented method comprising:
sensing or prompting a user to confirm the coupling of at least two drug containers to a respective one of at least two drug ports of a fluid guide system,
wherein the least two drug ports are configured to receive a fluid comprising at least one drug from at least one respective drug container,
wherein preferably, the computer-implemented method is performed by at least one electronic control unit of the medical device according to any one of aspects 11 to 16.

Aspect 20. The computer-implemented method according to aspect 19, comprising at least one of, several of or all of:
controlling a pump drive unit that is configured to drive a pump, wherein at least a part of the pump is implemented on the fluid guide system or wherein the pump is configured to generate the pumping force for a fluid flow within the fluid guide system,
prompting the user to begin a drug handling process,
performing a calculation to calculate a dose of the drug,
providing alerts and/or guidance to the user,
sensing the presence of at least one further container.

The further container may be an auxiliary container (e.g. comprising a diluent) or a further drug container (e.g. an infusion bag)

Aspect 21. A data processing device is provided, preferably a computer comprising a processor for carrying out the computer implemented methods according to aspect 19 or 20.

Aspect 22. A computer program product is provided comprising instructions which, when the program is executed by a computer, cause the computer to carry out any one of the computer implemented methods according to aspect 19 or 20.

Aspect 23. A computer readable medium is provided comprising instructions which, when executed by a computer, cause the computer to carry out any one of the computer implemented methods according to aspect 19 or 20.

Features mentioned in the description, e.g. in the first set of aspects or feature groups (FG) or in the appended claims for the fluid guide system, set, medical device, method, etc. may be applied to any one of the aspects of the second set of aspects and vice versa.

### 5. Features which may be Applied to Any Embodiment

A list of features, grouped in sublevels and containing variations and alternatives is presented in the following:

FG1. (Feature group) A single use consumable unit (e.g. fluid guide system), e.g. a cartridge or a flexible carrier may contain at least one, several of or all the fluid paths that contact the drug and the solution or liquid during preparation of the drug. The consumable unit may comprise at least one, several or all of the following features:

F1.1. (Feature) A feature to enable a fluid connection to drug containers, e.g. to drug vials

F1.1.1. Means or a feature to connect to a single drug container, e.g. vial at any time, e.g. valve portions, movable stage, and/or single fluid contact station,

F1.1.1.1. A drug container (e.g. vial) connection may be actuated within the device, e.g. to connect to an array of drug vials loaded by the user, e.g. using valves,

F1.1.1.2. The device may actuate drug containers, e.g. vials within the device:

F1.1.1.2.1. The device may move the drug container, e.g. a vial to insert the needle,

F1.1.1.2.2 The device may move the needle into the drug container, e.g. into the vial, see FIG. 18.

F1.1.2. Means or feature(s) to connect to several drug vials at the same time

F1.1.2.1. The drug container (e.g. vial) connections may be fixed; e.g. the user may load the drug containers (e.g. vials) into the correct array,

F1.1.2.2. The drug container connections may be actuated (e.g. using valves) by the device, e.g. in order to connect to an array of drug containers (e.g. vials) loaded e.g. by the user,

F1.1.3. A locking connection containing a single use snap fit to prevent re-use of consumable and/or vials and avoiding sterility/contamination risks, see e.g. FIG. 18.

F1.1.4. Means or unit to automatically remove the cap from the drug containers (e.g. vials) to simplify the user step.

F1.2. A feature for attaching the consumable to a diluent source, e.g. auxiliary container:

F1.2.1. This may incorporate the capability of the machine/device to accept either vial diluent sources or those attached through a carrier or other external container.

F1.3. A feature for connecting to an infusion bag,

F1.4. Compatibility of the consumable with the means for moving (FG2) and/or controlling (FG3) the fluid within the system,

F1.5. Internal fluid paths to contain and guide the fluid,

F1.5.1. Consisting of a set of connected tubes, e.g. flexible tubes or rigid tubes,

F1.5.2. Alternative embodiment to tubes and plastic casework, consisting of a multicompartment plastic, e.g. PVC (poly vinyl chloride) carrier, see e.g. FIG. 17. Heat sealing or another connection technique of specific areas of the carrier may form the channels for the fluid to move therein between.

F1.6. A casing or case, made out of a rigid material, e.g. a plastic material,

F1.7. External tubing and fittings such as Luer connectors or needles may be incorporated into the consumable unit,

F1.8. A filter to prevent particulates entering the system as air is drawn into the system,

F1.9. A lock and/or latch that prevents removal of the consumable from the system during reconstitution, e.g. a lock and/or latch interacting with cover member,

F1.10. The consumable unit may be packaged in a sterilizable sealed packet during transport and/or storage,

F1.11. Features to automate surface sterilization of drug containers, e.g. vials or drug containers may be used

F1.12. Features to limit user access to sterile areas of the consumable may be used, such as the drug container (e.g. vial) interface, e.g. to avoid contamination during handling of the consumable and/or of the drug containers.

FG2. Means or feature(s) of moving or unit for moving a controlled amount of fluid within the system, e.g. a pump interface:

F2.1. A peristaltic pump may be used within the device acting on tubing that may be part of the consumable,

F2.2. A diaphragm on the consumable may be actuated by mechanical or pneumatic means by the device to act as a diaphragm pump,

F2.3. Other positive displacement pumps may be used such as a Quantex pump, e.g. with the fluid contact elements within the consumable and/or the actuation means within the reusable device,

F2.4. In a system with a multi-compartment plastic carrier, e.g. PVC (Poly Vinyl Chloride) carrier consumable, pressure may be applied automatically to areas of the plastic carrier, e.g. PVC carrier, e.g. for example in a rolling motion, e.g. in order to provide a pumping function.

FG3. Means or feature(s) of controlling fluid access to different parts of the system:

F3.1. Valves to control the flow from the diluent source, e.g. auxiliary container to the drug containers, e.g. vials and then control the flow of the e.g. reconstituted drug solution from the drug containers, e.g. vials to a further drug container, e.g. to an IV carrier. Alternatively, mixing of liquid drugs may be performed by controlling the fluid flow from the drug containers, e.g. vials to a mixture carrier, e.g. an IV carrier.

F3.1.1. Number of connections/valves

F3.1.1.1. Each drug container, e.g. vial connection may have a valve to allow/block flow,

F3.1.1.2. Flow to several drug container, e.g. vial connections may be controlled by a single valve and there may be a means or unit to regulate the volume of fluid delivered to each individual drug container, e.g. vial., for example by configuring the flow path dimensions (e.g. diameters, lengths and/or interior surface features) so as to equalize resistance among flow paths Thus, a simple distribution element may be used, e.g. comprising at least on fluidical bifurcation, at least three bifurcations, etc.

F3.1.2. Valve Type

F3.1.2.1. Pinch valves actuated by mechanical or pneumatic means within the device may be used to compress e.g. part of the consumable's tubing to control flow without contacting the fluid. Alternatively, a diaphragm valve may be used.

F3.1.2.2. The consumable may contain diaphragm valves which may be actuated by mechanical or pneumatic means or features within the device to control flow without contacting the fluid.

F3.1.2.3. Allowing air to enter/leave vials to avoid vacuum formation, this may be an intrinsic feature of the drug containers, e.g. of vials. Alternatively, two needles may be used for access to one drug container, e.g. to a vial-one needle for liquid transport and one needle for venting.

FG 4. Means or feature(s) of ensuring an adequate reconstitution of lyophilized drugs or dilution of concentrated drugs and/or mixing of drugs:

F4.1. This may include mechanical agitation (automatic/or manually by e.g. a user), heat, fluid input and output control, etc.,

F4.1.1. Mechanical agitation-rotation,

F4.1.2. Mechanical agitation-vibration,

F4.1.3. Mechanical agitation-moving along a track, e.g. along a linear track, e.g. in opposite directions,

F4.1.4. Fluid agitation

F4.1.5. Let the user agitate it,

F4.1.6. Shake the whole device M,

F4.1.7. Shake the consumable once removed from the device, may use a replaceable cover member or a usage of no such cover member,

F4.1.8. Heated fluid, e.g. heated within the consumable or external of the consumable within the device.

F4.2. This may include features to avoid damaging sensitive drugs, e.g. special types of needles, etc.

FG5. A Computer system within the reusable device which may comprise:

F5.1. A facility for the user to begin reconstitution and/or mixing of drugs,

F5.2. A facility for calculation, including correct dose of the drug,

F5.3. A facility for providing alerts and guidance to the user of correct use steps and to avoid hazards,

F5.4. A sensing system for the detection of presence of the consumable unit, vial, diluent source and IV carrier, see e.g. FIG. 18,

F5.5. A facility to sense a digital signal from drug containers for programming purposes. This may incorporate RFID (Radio Frequency Identification) and/or optical (barcode) methods.

F5.6. A facility to download the user's treatment plan from the cloud,

F5.7. A facility to record what was prepared and/or upload to the cloud.

FG6. Design features to improve quality of life using the system may include at least one of:

F6.1. Parts of the device folding to allow for a small storage volume when not in use, see e.g. FIG. 11, hinges.

F6.2. A location to place an infusion bag, e.g. FIG. 11, support portion.

FG7. Flexibility with the number of drug containers, e.g. vials:

F7.1. Accept consumable waste—The device and the consumable may be designed to take a maximum number of drug containers, e.g. vials and only partially filled for smaller number of drug containers, e.g. vial treatments, see e.g. FIG. 15,

F7.2. Consumable SKUs (stock keeping unit)—The device may be designed to take a maximum number of drug containers, e.g. vials. A set of different types of consumables may be available with different numbers of drug ports, e.g. of vial inputs to reduce the size of the consumable and to reduce waste of consumable for smaller number of drug containers, e.g. vial treatments, see e.g. FIG. 15, dashed lines,

F7.3. Modular consumable—The device may have a small number of basic drug container inputs, e.g. vial inputs. Adapters may be used to expand the number of inputs to allow more drug containers, e.g. vials to be connected to a single basic input. Additionally or alternatively, the adapters may be used to regulate the volume between attached drug containers, e.g. vials, see e.g. FIG. 16 and remarks with regard to feature F3.1.1.2.

F7.4. Consumable and/or the device may have single vial connection, that is used on several vials/treatment, e.g. a movable stage, 1D (dimensional), 2D or even 3D (e.g. for needle insertion).

FG8. A means or feature(s) of or a unit for automated validation of adequately complete reconstitution of drugs:

F8.1. User validation—drug containers, e.g. vials may be visible and the device may ask a user to confirm success of reconstitution, e.g. drug containers have to be empty at the end of the drug handling process.

F8.2. Non-contact visual or optical—i.e. optical sensing or UV (ultra violet) and/or vis (visible), e.g. within the range of 400 nm (nanometer) to 750 nm,

F8.3. Contact sensing—The pH (potential of hydrogen) value may be sensed or other physical parameter, e.g. within the drug container, e.g. vial.

Thus according to an embodiment an auto-reconstitution machine is provided to automate e.g. lyophilized or concentrated drug product reconstitution, dosing and transfer to an intravenous IV carrier.

The embodiments are intended to be used e.g. as part of a dilution or reconstitution process, for example in the preparation of multiple similar vials of drug for an IV infusion.

The embodiments may be used to automate the procedure where multiple vials are currently required to be diluted or reconstituted manually by the user, with several steps and using several additional consumables.

In one embodiment, a machine may automate drug preparation. The user may assemble the fluid containers, and the machine/device may do many of the currently manual steps-including dosing and/or reconstitution and/or IV carrier filling.

KEY DRIVERS may be at least one, several or all of: .
TIME SAVINGS: Filling multiple vials or other drug containers quickly rather than sequentially via manual manipulation substantially reduces time required for drug preparation; and/or
SAFETY: Removes needle stick risk preventing user access to vial access spikes, and/or
STERILITY: Fewer manual handling steps may reduce the risk of contamination, and/or
EASE OF USE: Fluid transfer and dosing may be automated, reducing the time, effort and dexterity effort of the user, and/or
EFFICACY: Removes manual user errors in dosing by automating the process, and/or
AVOID DRUG DAMAGE: Controls diluent flow to avoid jetting onto the drug, and/or
MINIMISE WASTE: Removes the need for transfer syringes and/or needles and associated packaging, replacing them with a single consumable part.

A machine to automate drug preparation is proposed—making the process simpler, safer and quicker for the user. There are several possible embodiments, but they all may comprise or may consist of a reusable portion of the system and a single-use consumable and may share at least one of the following key features:

### Automated Fluid Control

Fluid system, comprising electromechanical pump(s) and valves within the reusable device, to transfer the correct amount of diluent to the drug containers, e.g. vials, reconstitute/dilute and transfer the drug solution to the IV carrier or another appropriate drug container, e.g. for drug mixing.

### Sterile Consumbale

Loaded into the device before each use, this may be the only part that contacts the fluid. It may contain interfaces to the drug vials, diluent, IV carrier and the fluid control system.

### User Interface

A user interface on the device may guide the user through the loading steps and may prompt the user to check and confirm correct setup and/or the correct reconstitution or another drug handling procedure.

Embodiment—Auto-Recon System with a consumable containing multiple fixed vial connections
System may consist of a reusable device and a single use consumable, and/or
The consumable may consist of or may comprise tubes and/or plastic moldings. It may be sterilized before use and may contain all the system's fluid paths, and/or
The consumable may have multiple fixed drug vial connections, fluid access to each may be controlled by a valve or valve portion on the reusable device.

The reusable device may comprise at least one, several or all of:
A reconstitution and/or dispensing pump, and/or
A fluid control system, e.g. ECU, and/or
An integrated user interface (UI), and/or
The device may be powered by the mains, e.g. the electrical grid of a utility company or by a battery, e.g. by a rechargeable battery or by a non-rechargeable battery, and/or
The consumable may be quick and/or easy to load, and/or
The device may comprise smooth surfaces which are easy to clean, and/or
The device may be “foldable”, e.g. to closed storage configuration.

The consumable/removable fluid guide system may comprise at least one, several or all of:
A single-use sterile assembly—tubes and/or plastic moldings, and/or
The fluid guide system may be the only component that contacts the fluid, and/or
Multiple vial adapters, diluent connection, IV (intravenous) carrier connection and/or pump interface and valve interface, and/or
One step loading of the fluid guide system into the machine/device may be possible.

The making and using of the presently preferred embodiments are discussed in detail below. It should be appreciated, however, that the present disclosure provides many applicable concepts that can be embodied in a wide variety of specific contexts. The specific embodiments discussed are merely illustrative of specific ways to make and use the disclosed concepts, and do not limit the scope of the claims.

Moreover, same reference numerals refer to same technical features if not stated otherwise. As far as “may” is used in this application it means the possibility of doing so as well as the actual technical implementation. The present concepts of the present disclosure will be described with respect to preferred embodiments below in a more specific context namely a drug reconstitution medical device and corresponding fluid guide system, e.g. cartridge system or flexible carrier system. The disclosed concepts may also be applied, however, to other situations and/or arrangements as well, e.g. to combined drug handling devices and infusion or injection devices, e.g. the removable fluid guide system may comprise an injection or infusion mechanism, to auto-injectors or to other medical devices.

The foregoing has outlined rather broadly the features and technical advantages of embodiments of the present disclosure. Additional features and advantages of embodiments of the present disclosure will be described hereinafter, e.g. of the subject-matter of dependent claims. It should be appreciated by those skilled in the art that the conception and specific embodiments disclosed may be readily utilized as a basis for modifying or designing other structures or processes for realizing concepts which have the same or similar purposes as the concepts specifically discussed herein. It should also be recognized by those skilled in the art that equivalent constructions do not depart from the spirit and scope of the disclosure, such as defined in the appended claims.

### BRIEF DESCRIPTION OF THE DRAWINGS

For a more complete understanding of the presently disclosed concepts and the advantages thereof, reference is now made to the following description in conjunction with the accompanying drawings. The drawings are not drawn to scale. In the drawings the following is illustrated in:

FIG. 1 a first configuration of a medical device comprising a fluid guide system, e.g. of a cartridge system or of a flexible carrier system,

FIG. 2 a second configuration of a medical device comprising a fluid guide system, wherein the fluid guide system comprises more components compared to the first configuration,

FIG. 3 a third configuration of a medical device comprising a fluid guide system, wherein the removable fluid guide system comprises less components compared to the first configuration,

FIG. 4 a medical device comprising a fluid guide system, preferably in a pre-use state,

FIGS. 5 to 9 the medical device of FIG. 4 in different operation states of drug handling, e.g. priming, dilution/reconstitution of a first drug container, e.g. vial, of a second drug container, etc., flushing and transfer of the reconstituted drugs to a further drug container,

FIG. 10 a medical device comprising a fluid guide system comprising a flow divider,

FIG. 11 a reusable medical device in a state with a closed cover,

FIG. 12 the reusable medical device in a state with an open cover,

FIG. 13 a perspective view to an embodiment of a fluid guide system usable together with the medical device,

FIG. 14A a top view to the fluid guide system according to FIG. 13,

FIG. 14B a variant of the embodiment of FIG. 14A,

FIG. 15 an embodiment of a fluid guide system of a set of fluid guide systems,

FIG. 16A an alternative embodiment of a basis fluid guide system and a modular flow divider,

FIG. 16B a detail of a module of the embodiment as illustrated in FIG. 16A,

FIG. 17 a plastic carrier fluid guide system, and in

FIG. 18 an embodiment of a flow control portion and of a drug port, e.g. of a vial port.

### DESCRIPTION OF EXEMPLARY EMBODIMENTS

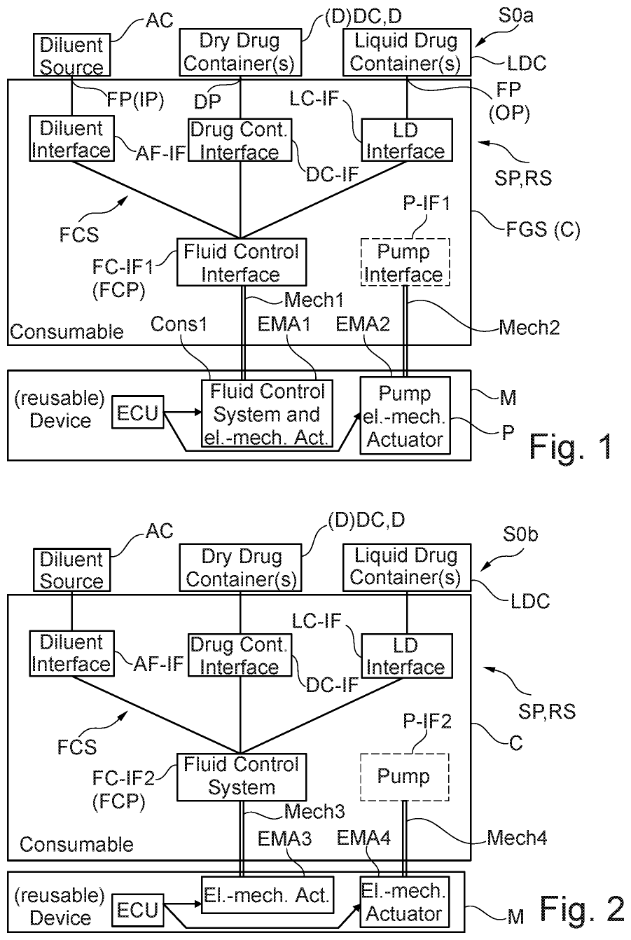

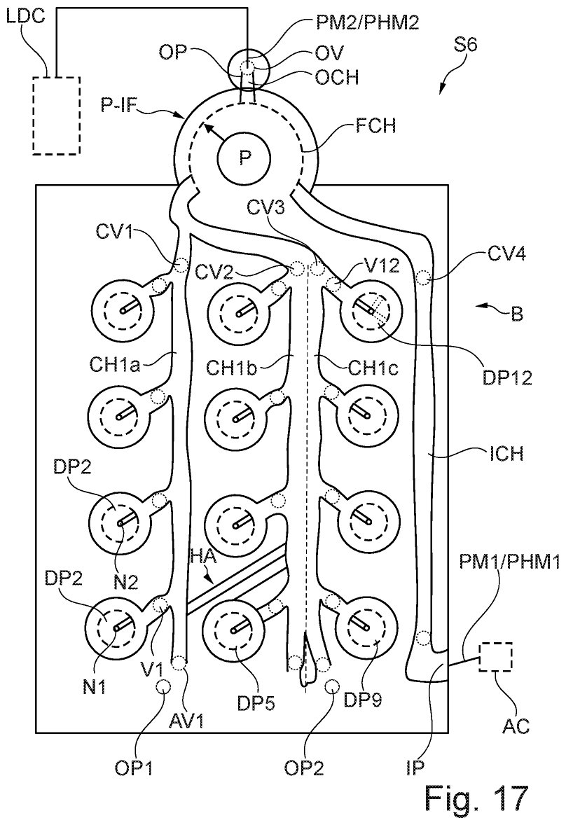

### FIG. 1 Illustrates a First Configuration of a Medical Device Comprising a Fluid Guide System FGS, e.g. a cartridge system comprising a Cartridge C or a Flexible Carrier System, see e.g. FIG. 17

In FIGS. 1 to 3, single lines represent a fluid connection. Further, double lines represent a mechanical connection.

A reconstitution system S0a may comprise:
The fluid guide system FGS, and
A medical device M, e.g. a machine.

Firstly, the fluid guide system FGS is described. The medical device M is described below in more detail. However, several drug containers may be supported by the device (machine) M, e.g.:
An auxiliary container AC, e.g. comprising a diluent, and/or
A liquid drug container LDC (may comprise already a second auxiliary liquid).

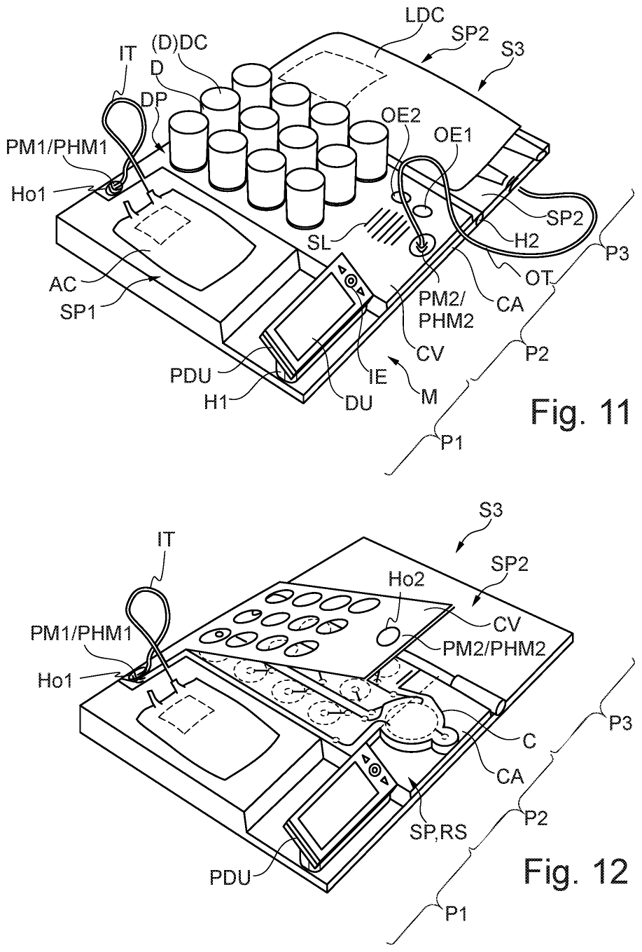

The auxiliary container AC may have mechanical/physical contact to machine/device M, e.g. to an auxiliary support portion SP1, see e.g. FIG. 11. The auxiliary container AC may be optional, e.g. if a liquid drug container LDC has the function of storing a diluent and of storing the end product of the process. Thus, in the case of using no auxiliary container, the liquid drug container may have only one compartment, e.g. for the diluent and for the product of the mixing and/or reconstitution, or two compartments, e.g. one for the diluent and one for the product of the mixing and/or reconstitution. The auxiliary container AC may be a flexible carrier, e.g. a plastic carrier. The auxiliary container AC may comprise a diluent or another liquid, e.g. WFI (water for injection).

According to another embodiment, auxiliary drug containers, e.g. vials may be placed on the fluid guide system FGS as well. Thus, also in this embodiment, auxiliary support portion SP1 would not be necessary.

A liquid drug container LDC may have mechanical/physical contact to machine/device M, e.g. to an LDC support portion SP2 of medical device M, see e.g. FIG. 11. The liquid drug container LDC may be a flexible carrier, e.g. a plastic carrier, preferably an infusion bag. The liquid drug container LDC may be empty at the beginning. Alternatively, the liquid drug container LDC may comprise an auxiliary liquid at the beginning, e.g. a saline solution. At the beginning no drugs may be comprised within the pre-filled liquid drug container LDC. At the end of the drug handling process, the liquid drug container LDC may comprise the drugs which were formerly within the drug containers DC, DDC, e.g. preferably dry drug containers.

Moreover, at least one dry drug container DDC or several dry drug containers DDC may be supported by the fluid guide system FGS, e.g. cartridge or flexible carrier and/or by a cover member of medical device M. The at least one dry drug container DDC, e.g. at least one vial may comprise at least one type of drug D or at least several types of drugs, especially of lyophilized drug(s). The at least one dry drug container DDC may be in mechanical/physical contact to machine M, e.g. to a cover member CV (see e.g. FIGS. 11 and 12) and/or to fluid guide system FGS, e.g. a cartridge C or a carrier B.

In other embodiments, the drug containers DC may comprise liquid drug formulations at the beginning. The drug containers may be named therefore as drug containers DC.

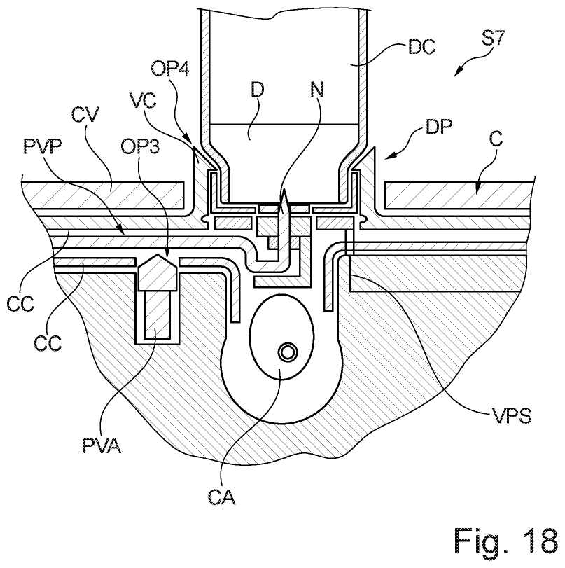

The fluid (e.g.) liquid guide system FGS may comprise:
A fluid port FP, e.g. an inflow port IP, and/or
At least one drug port DP, e.g. a plurality of drug ports DP, see e.g. FIG. 18, and/or
An outflow port OP, optionally fluid port FP may also have the function of an outflow port.

During the preparation of the medical device M, the fluid port FP may be coupled to the auxiliary container AC (optionally, also possible to use LDC only as mentioned above). An auxiliary fluid interface A-IF may provide mechanical/physical contact of the auxiliary container AC to device M and/or a fluid connection to fluid guide system FGS. Thus, a Luer-Lock connector and/or another connector as well as a flexible tube may be used to connect an outflow port of auxiliary container AC with fluid port FP.

During the preparation of the medical device M, the at least one drug port(s) DP may be coupled to the at least one dry drug container DDC, e.g. to respective vials. A drug container interface DC-IF may provide fluidically and mechanically contact between dry drug containers DDC and the fluid guide system FGS. Optionally, the drug container interface DC-IF may provide a cleaning function (sterilizing) and/or a moving function for needles or a fixing function for the dry drug containers DDC. An example of a drug port DP is described below in more detail, see FIG. 18 and corresponding description thereof.

During the preparation of the medical device M, the liquid drug container LDC (e.g. may comprise already second auxiliary liquid) may be coupled to the outflow port OP or to fluid port FP. A liquid drug container interface LC-IF may provide fluidically and mechanically contact between liquid drug container LDC and the fluid guide system FGS, e.g. a cartridge C or a carrier B. Thus, a Luer-Lock connector and/or another connector as well as a flexible tube may be used to connect a port of the liquid drug container LDC with the output port OP or optionally with the fluid port FP.

The fluid guide system FGS may comprise a fluid control interface FC-IF1, e.g. at least one tube portion that is configured to interact with a pinch valve, at least membrane portion configured to interact with a membrane valve actuator or other fluid interaction portions. These portions are flow control portions FCP. In a first operation mode M1 (flow-through), flow through the respective flow control portions FCP may be enabled, e.g. by a pinch valve portion PVP, see FIG. 18. In a second operation mode M2 flow through the respective flow control portions FCP may be blocked, e.g. by the pinch valve portion PVP. Thus, the complete valve or almost the complete valve may be arranged within the device M.

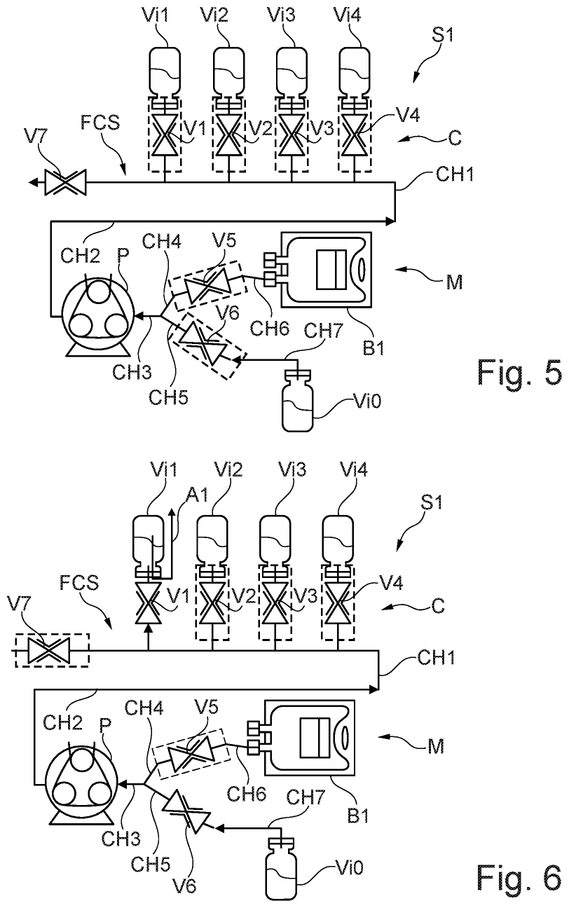

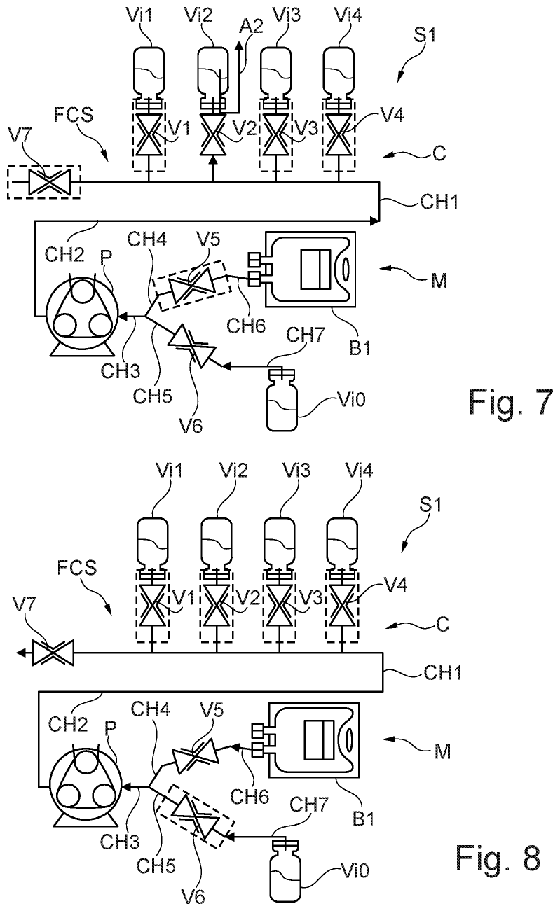

The fluid channel system FCS may comprise a fluid channel system FCS that provides at least one of, several of or all of the following:
A fluid connection between the auxiliary interface AF-IF (e.g. diluent interface) and the drug container interface DC-IF, preferably including at least a portion of the fluid control interface FC-IF1 in order to enable at least one switchable fluid connection between these two interfaces AF-IF and DC-IF.
A fluid connection between the dry drug container interface DC-IF and the liquid drug container interface LC-IF, preferably including at least a portion of the fluid control interface FC-IF1 in order to enable at least one switchable fluid connection between these two interfaces DC-IF and LC-IF.
Optionally, other fluid connections, e.g. for priming, see FIG. 5 and/or for venting/flushing, see FIG. 7, preferably using again at least a portion of the fluid control interface FC-IF1 in order to enable at least one switchable fluid connections or at least two switchable fluid connections.

An optional pump interface P-IF1 (portion) may be used that is described in more detail below, see e.g. pump interfaces P-IF. Alternatively, all parts of the pump P may be arranged within the device M.

No contact of reusable pump parts and the liquid within fluid guide system FGS may be established. Usage of a positive displacement pump may reduce damage to cells, e.g. blood cells or to biomolecules, special drugs, etc. However, depending on the kind of drugs, usage of negative pressure pumps may also be considered. Only the tubing (e.g. for a peristaltic pump or the diaphragm (e.g. for a diaphragm pump may be part of the consumable (FGS), e.g. of a cartridge or of a flexible carrier. The tube/diaphragm interface and the electromechanical actuator that provides the motion to move the fluid may be arranged within the reusable device M.

The support portion SP/receiving space RS of the device M may be a closed or closable space, e.g. closable by a cover member CV, see FIGS. 11 and 12. Alternatively, the support portion SP/receiving space may be open to at least one side, e.g. to the upper side.

The device M may comprise:
An electronic control unit ECU, and/or
A fluid control system ConS1, and/or
A pump P.

The electronic control unit ECU may control, e.g. the fluid control system ConS1 and/or the pump P. The electronic control unit ECU may comprise a processor or a finite state machine without a processor.

The fluid control system ConS1 may comprise at least one valve configured to interact with a respective tubing portion or valve membrane/diaphragm portion of the fluid guide system FGS. The valve may comprise e.g. an electromagnetic coil and an actuator element (e.g. pin), a linear motor moving a pin, etc. Alternatively, pneumatically or hydraulically operated valves may be used.

The electro mechanical actuator(s) EMA1 of the fluid control system ConS1 may form a mechanical interface Mech1, e.g. a detachable connection to the tubing portion or other valve portion of the fluid guide system FGS.

The pump P may be a pump of one of the types mentioned above, e.g. a peristaltic pump. An electro mechanical actuator EMA2 may provide a mechanical interface Mech2, e.g. a detachable connection to the tubing/pumping membrane/diaphragm of the fluid guide system. Thus interaction between e.g. at least one wheel, e.g. at least one friction wheel of a peristaltic pump and the respective tubing of the fluid guide system may be provided by the mechanical interface Mech2.

In this embodiment of system S0a all components that contact the fluid may be within the consumable (fluid guide system FGS) and non-contact pumping and non-contact fluid control technologies may be used. The fluid control system ConS1 may be a set of pinch or diaphragm valves. The fluid control interface FC-IF1 such as the tubing that is pinched (pinch valve) or the diaphragm that is stretched (diaphragm valve) may be part of the consumable, e.g. of the fluid guide system FGS. The electromechanical actuator and a mechanism that converts the actuator motion into a motion to open and close the valves may be within the reusable device, e.g. within medical device M.

The pump P could be a peristaltic or a diaphragm pump. The tubing (peristaltic) or the diaphragm (diaphragm pump) may be part of the pump interface P-IF1 of the consumable, e.g. of the fluid guide system FGS. The tube/diaphragm contact parts (e.g. counter part of the pump interface) and the electromechanical actuator that provides the motion to move the fluid may be within the reusable device M. Other pump types may be used as well, e.g. positive displacement pumps.

### FIG. 2 Illustrates a Second Configuration, e.g. System S0b of a Medical Device Comprising a Fluid Guide System FGS, Wherein the Removable Fluid Guide System FGS Comprises More Components Compared to the First Configuration

However, there are many similar or identical elements that are not described in detail again. Only the differences between the system S0a and the system S0b are described in the following.

The fluid control interface FC-IF2 may comprise parts of the valve, e.g. parts of a pinch valve, membrane valve, a small rotatable valve, e.g. comprising plastic, or of other interaction areas. Thus, exemplary, the valve(s) may be arranged within the fluid guide system FGS. However, the mechanical actuator may still be within the reusable device M as well as a driving unit for the actuator. The actuator on the reusable part may be e.g. pneumatic, mechanical or electromechanical.

An optional pump interface P-IF2 (portion) may comprise more parts of the pump if compared with system S0a, e.g. the at least one, at least two or at least three wheels of a peristaltic pump or of another tape of pump, e.g. positive displacement pump.

A mechanical interface Mech3 may establish a detachable connection between the electro mechanical actuator EMA3 and the valve parts arranged in the consumable (FGS). Mechanical actuator EMA3 may comprise e.g. two pinch elements per valve.

A mechanical interface Mech4 may establish a detachable connection between an electro mechanical actuator EMA4 and the pump parts arranged on consumable (FGS). Mechanical actuator EMA4 may be a tooth wheel comprising outer teeth interacting with inner teeth of a tooth wheel coupled e.g. to the three wheels of a peristaltic pump.

The description of FIG. 1 remains valid for the other items of FIG. 2, e.g. auxiliary container AC, dry drug container DDC, liquid drug container LDC, auxiliary interface AC-IF, drug container interface DC-IF, liquid drug container interface LC-IF, electronic control unit ECU, etc.

In this alternative embodiment, i.e. system S0b, all components that contact the fluid may be arranged within the consumable formed by the fluid guide system FGS, e.g. a cartridge C or a carrier B. All components of the fluid control system FCS may be within the consumable apart from e.g. the electromechanical actuator EMA3 that converts electrical energy into a rotary or linear motion. The electromechanical actuator EMA3 may be arranged in the reusable device M and/or the respective driving unit.

All the pump P components may be arranged within the consumable apart from the electromechanical actuator EMA4 that converts electrical energy into a rotary or linear motion which may be arranged in the reusable device M.

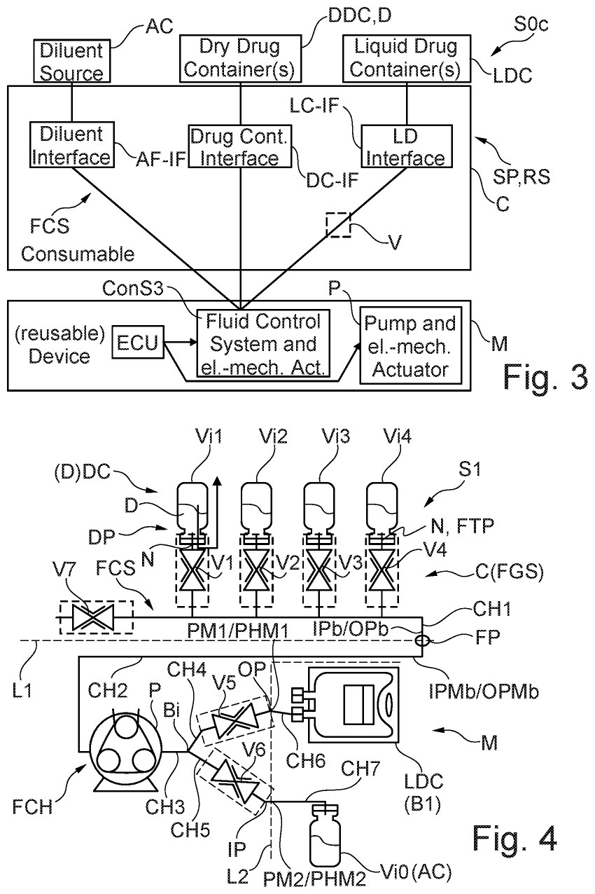

### FIG. 3 Illustrates a Third Configuration, e.g. System S0c of a Medical Device Comprising a Fluid Guide System FGS Wherein the Removable Fluid Guide System FGS Comprises Less Components Compared to the First Configuration

However, there are many similar or identical elements that are not described in detail again. Only the differences between systems S0a and S0c are described in the following.

A fluid control system ConS3, e.g. electromagnetic coils and actuator, linear motor, etc. may be arranged completely within reusable device M. Alternatively, pneumatically or hydraulically operated valves may be used, e.g. also arranged completely within reusable device M.

The fluid channel system FCS of the fluid guide system FGS may not comprise any parts of valves, valve portions, of a pump or a pump portion. On the contrary, there may be additional fluid ports between the fluid channel system FCS of the fluid guide system FGS and the reusable device M, e.g. coupled only to the fluid control system ConS3 and/or to the pump P and the electromechanical pump actuator. An optional fluid connection may connect the fluid control system ConS3 and the pump P and the electromechanical pump actuator. Thus, all additional ports of the fluid channel system FCS may be connected or may be connectable only to the fluid control system ConS3.

In system S0c, there may be no mechanical connections between fluid guide system FGS and reusable medical device M.

According to another embodiment of system S0c, at least one optional fluid valve V or valve portion may still be arranged within fluid guide system FGS.

The description of FIG. 1 remains valid for the other items of FIG. 3, e.g. auxiliary container AC, dry drug container DDC, liquid drug container LDC, auxiliary interface AC-IF, drug container interface DC-IF, liquid drug container interface LC-IF, electronic control unit ECU, etc.

In this alternative embodiment, i.e. system S0c the pumping and fluid control system ConsS3 may be within the reusable device M. The consumable, e.g. a cartridge C or a carrier may connect these systems to the containers, e.g. diluent, vial(s) and IV carrier.

Given the fluids contact the fluid control system ConS3 and pump P within the reusable device M, these systems may require cleaning and/or sterilizing after and/or before each use.

### FIG. 4 Illustrates a Medical Device M Comprising a Fluid Guide System FGS, Preferably in a Pre-Use State

A system S1, e.g. a reconstitution system may comprise:
A fluid guide system FGS, e.g. according to one of the embodiments of systems S0a, S0b described above, preferably comprising a cartridge C or a plastic carrier B, see e.g. FIG. 17, and
A medical device M, e.g. a reconstitution device or another drug handling device.

The border between device M and the fluid guide system, e.g. a cartridge C or a carrier B may be different in different embodiments, see e.g. only line L1 or only line L2.

In the embodiment for which line L1 is valid, the following parts may be arranged in the fluid guide system FGS, see e.g. FIG. 14B:
A fluid channel CH1, and
Valves V1 to V4 and V7.

In the embodiment for which line L1 is valid, the following parts may be arranged in or on device M, see e.g. FIG. 14B:
A channel CH2, e.g. connected to or connectable to the channel CH1,
A pump P, e.g. a peristaltic pump or another type of pump,
Channels CH3, CH4 and CH5 e.g. arranged fluidically between pump P and an auxiliary container AC and/or a liquid drug container LDC,
Valves V5 and V6, e.g. connected to channels CH3 and CH4,
Channels CH6 and CH7,
At least one auxiliary container AC, e.g. a vial Vi0, and
A liquid drug container LDC, e.g. an IV infusion bag.

The fluid guide system FGS may comprise only one fluid port or several fluid ports which are used as input fluid port(s) IPb and output fluid port(s) OPb, i.e. as common input/output port IP/OP. Thus, the device M may also comprise only one common port IPMb and OPMb coupled to (fluidically and/or mechanically) the common fluid port IPb/OPb.

In the embodiment for which line L2 is valid, the following parts may be arranged in the fluid guide system FGS, see e.g. FIG. 14A:
The fluid channels CH1 to CH5,
All the valves V1 to V7, and
The pump P.

In the embodiment for which line L2 is valid, the following parts may be arranged in or on device M, see e.g. FIG. 14A:
Channels CH6 and CH7, and
The at least one auxiliary container AC, e.g. a vial Vi0, and
The liquid drug container LDC, e.g. an IV infusion bag.

In the embodiment for which line L2 is valid, the fluid guide system FGS may comprise at least one input fluid port IP or several fluid ports IP which are used as flow input ports only. The fluid guide system FGS may comprise at least one output port OP or several output ports OP which are used as flow outputs only.

Thus, the fluid guide system FGS may comprise at least one first port coupling element PHM1 which is coupled or coupleable (fluidically and/or mechanically) to the input port IP of the fluid guide system FGS. The first fluid port may be coupled fluidically to the auxiliary container AC, e.g. via a first tube. The first fluid port may be an end of the first tube IT or a first tube adapter coupled fluidically to the end of the first tube. The first tube adapter may be disposable, e.g. together with the first tube IT and/or with the fluid guide system FGS.

Moreover, the fluid guide system FGS may comprise at least one second port coupling element/member PHM2 coupled or coupleable (fluidically and/or mechanically) to the output port OP of the fluid guide system FGS. The second fluid port may be coupled fluidically to the liquid drug container (L) DC, e.g. using a second tube OT. The second fluid port may be an end of the second tube or a second tube adapter coupled fluidically to the end of the second tube. The second tube adapter may be disposable, e.g. together with the second tube OT and/or with the fluid guide system FGS.

The ports, e.g. PHM1, PHM2 to connect to the diluent bag or other container and to the IV bag or other container may be on the consumable. These ports may therefore be disposed of after each treatment. Thus, no fluid would contact the cover CV or any other part of the reusable device. Cleaning steps may be avoided thereby without lowering sterility.

Thus, it may be preferred, that the connection between the consumable fluid path and the diluent source/liquid drug containers may be part of the consumable.

For the embodiment for which line L2 is valid, there may be no fluid port FP and/or no ports IPb/OPb and IPMb/OPMb.

In the embodiment of system S1, four dry drug containers DDC may be used as a maximum, e.g. vials Vi1 to Vi4. The dry drug container DDC may be coupled (mechanically and/or fluidically) to a plurality of respective drug ports DP, e.g. to four drug ports in the embodiment of system S1. The drug ports DP may comprise movable needles N, see e.g. FIG. 18 or stationary needles N, i.e. needles N which cannot be moved relative to a cartridge C and/or relative to device M if the fluid guide system FGS is received within or on the support portion SP/receiving space RS. A different number of drug ports DP may be used as well, e.g. in the range of 1 to 50. Instead of needles N, other types of fluid transmitting portions FTP may be used as well, e.g. cannulas without sharp tips.

Moreover, each drug port DP may be fluidically coupled or coupleable to a respective valve V1 to V4 or to a respective valve portion of valves V1 to V4. FIG. 18 also illustrates an example of a pinch valve portion PVP.

Drug containers DDC may be coupled mechanically directly to fluid guide system FGS and indirectly to device M when fluid guide system FGS is received within or on the support portion SP/receiving space RS. Alternatively, drug containers DDC may be coupled mechanically to device M, e.g. to cover member CV that is described below.

Drug ports DP may comprise respective needles N. Each needle N may be fluidically connected to a respective valve V1 to V4. The other side of a respective valve V1 to V4 may be fluidically connected to channel CH1.

One end of the channel CH1 may be fluidically connected to the valve V7. The other end of the channel CH1 may be fluidically connected to the fluid port FP (variant according to line L1) or to the channel CH2 (variant according to line L2).

One end of the channel CH2 may be fluidically connected to the fluid port FP (variant according to line L1) or to the channel CH1 (variant according to line L2). The other end of channel CH1 may be fluidically connected to one end of a fluid chamber FCH or a portion which is pressed and released by pump P. The other end of fluid chamber FCH or of the portion may be fluidically connected to one end of channel CH3.

The other end of channel CH3 may be fluidically connected to a respective end of channel CH4 and of channel CH5. Thus, there may be a bifurcation Bi of channel CH3 to channels CH4 and CH5 in one flow direction, e.g. for flow coming from the pump P via channel CH3. A merging portion of channels CH4 and CH5 may be formed at the same location at which the bifurcation Bi is arranged in the opposite flow direction, e.g. to pump P.

The other end of the channel CH4 is fluidically connected to one side of the valve V5 (or of the valve portion V5). The other side of the valve V5 (or of the valve portion V5) may be fluidically connected to the channel CH6 which may lead to the liquid drug container LDC, e.g. an infusion bag or to a plurality of liquid drug container LDC.

The other end of the channel CH5 is fluidically connected to one side of the valve V6 (or of the valve portion V6). The other side of the valve V6 (or of the valve portion V6) may be fluidically connected to the channel CH7 which may lead to the auxiliary container AC, e.g. a flexible carrier or a vial Vi0. Alternatively, several auxiliary containers AC may be used.

Thus, the fluid channel system FCS of the fluid guide system FGS may comprise several fluid channels, e.g. needles or needle portions, channels CH1 and/or channel CH2, etc. Some of the channels may be fluidically separated from other channels in one operation mode of a respective valve V or of respective valves V. Moreover, the same channels may be fluidically connected to the same channel(s) in another operation mode of the same valves V, e.g. a channel between valve V1 and vial Vi1 relative to channel CH1, etc.

In the following the operation of system S1 is described in detail. A dashed rectangle around a valve V illustrates the closed state of this valve. If there is no dashed rectangle around a valve V, this illustrates the open state of this valve V. The valves may be actuated by electromechanical or other actuators. Driving units may drive the actuators. The driving units may be controlled by the electronic control unit ECU.

### Step St0: Coupling of the Vials to the Drug Ports

At the beginning, the vials Vi1 to Vi4 may be coupled to the drug ports DP, e.g. fluidically via e.g. needles and/or mechanically, e.g. via mechanical elements as mentioned in the introductory part and/or below, see e.g. FIG. 18, vial engagement clips VC. The coupling of the vials Vi1 to Vi4 to the drug ports may be done manually. At the end of step S0, all four vials Vi1 to Vi4 are mechanically and/or fluidically coupled to the respective drug port. Thus, the following steps may be performed without further coupling of at least one vial to a drug port. This may allow fast performance of the reconstitution steps or of other drug handling steps.

### Step St1: Pre-Use Step or Pre-Use Configuration

All valves V1 to V7 are closed. Thus, there is no fluid connection between e.g. auxiliary container AC and channels CH1/CH2. Moreover, there are no fluid connections between dry drug containers DDC (which may comprise lyophilized drugs D) and channel(s) CH1/CH2. Channel CH1 is not fluidically connected to the environment because valve V7 is closed. The pump P may be in a switched off state. Moreover, valve V5 may be in a closed state thereby preventing that fluid flows out of liquid drug container LDC which may comprise a saline solution at the beginning of the reconstitution process which is described in the following.

FIGS. 5 to 9 illustrate the medical device M of FIG. 4 in different operation states of drug handling, e.g. priming, dilution/reconstitution of a first drug container, e.g. vial, of a second drug container, etc., flushing and transfer of the reconstituted drugs to a further drug container (e.g. IV carrier).

### FIG. 5 Illustrates a Step St2: Priming Step or Priming Configuration

There may be the following valve states:
Valves V1 to V4 are still closed,
Valve V5 remains closed, and
Valves V6 and V7 are open.

The pump P may be switched on by the electronic control unit ECU. Thus, fluid is pumped from fluid source, e.g. diluent in auxiliary container AC, e.g. vial Vi0 through the pump P or through a fluid chamber FCH of pump P to prime the tubing, e.g. channels CH1 and CH2 with fluid, e.g. a liquid fluid, preferably with a diluent. Air is pushed out through valve V7 in order to vent/drain the fluid channel system FCS. The fluid flow that is used for flushing may be stopped when all air is flushed through system S1, i.e. there is no air or only small residues of air within system S1.

### FIG. 6 Illustrates a Step St3: Dilution/Reconstitution Vial Vi1 (or Other Drug Container DC) or Dilution Configuration

There may be the following valve states:
Valve V1 open,
Valves V2 to V4 are still closed,
Valve V5 remains closed,
Valve V6 remains open, and
Valve V7 is closed.

Using pump P (controlled by the ECU), fluid is pumped out from auxiliary container AC, e.g. vial Vi0 to drug Vial Vi1, e.g. through channels CH1 and CH2. The necessary fluid (e.g. liquid) volume may be pumped in a controlled manner (ECU) by e.g. a positive displacement pump, e.g. pump P. Air may be vented from drug container DC1, e.g. from vial Vi1 by a hydrophobic vent which may be part of the consumable to vial connection which may allow air only to freely escape/enter, see arrow A1. Alternatively, there may be a negative pressure within vials Vi1 to Vi4 or other drug containers, e.g. mitigating the venting issue.

According to another embodiment, the needle may have two channels or two needles may be used per vial or per other drug container DC.

If the vial or the other drug contained DC is connected to the atmosphere, a filter may be used to lower the risk of contamination, e.g. an aseptic particulate filter.

Pumping may be stopped at the end of step St3, e.g. when enough liquid for reconstitution is pumped into vial Vi1 or another first drug container DC.

### FIG. 7 Illustrates a Step St4a (Similar to Steps St4b to St4x): Dilution/Reconstitution of Vial Vi2 to x, e.g. Vial Vi4.

There may be the following valve states:
Valve V1 is closed,
Valve V2 is open,
Valves V3 and V4 are still closed,
Valve V5 remains closed,
Valve V6 remains open, and
Valve V7 is closed.

Fluid may be pumped from the liquid source, e.g. the diluent source vial Vi0 to drug Vial 2 or another second drug container. The liquid volume may be pumped controlled using the ECU and e.g. a positive displacement pump. Air may be vented from drug vial Vi2 or another second drug container by a hydrophilic vent which may be part of the consumable to vial connection which allows air only to freely escape/enter, see arrow A2. Thereafter, pump P may be stopped or switched off by the ECU.

This process may be repeated until all vials Vi1 to Vi4 or other drug containers DC are filled to an appropriate level, e.g. with the diluent.

At the end the valve of the last drug port DC may be closed, e.g. valve V4. Valve V6 may also be closed.

An optional intermediate reconstitution step is not illustrated in a separate figure. During reconstitution now fluid flows may be established, e.g. no valves or valve portions of valves V1 to V7 may be operated by the ECU. Reconstitution may last a specific time, e.g. in the range of 1 minute to 1 hour or in the range of 5 minutes to 50 minutes to give only two possible examples. Reconstitution may be supported by additional measures, e.g. as mentioned in this document and/or in order to shorten the time necessary for complete reconstitution of drug(s) D.

Thus, there may be a step here where the machine M may do not anything actively, but essentially pauses to give time for the reconstitution to occur. One variant may be that the machine M repeatedly pushes fluid in and out of the vial to encourage/speed up reconstitution.

Additional measures may comprise:
Heating or warming of drug vials Vi1 to Vi4 or of other drug containers DC, and/or
Gentle shaking of device M, e.g. manually or automatically, e.g. using a shaking device available in the market, and/or
Other measures as mentioned above and/or below, etc.

### FIG. 8 Illustrates a Step St5: Flush(ing) or Flush Configuration

At the end of the reconstitution phase, the following valve states may be established by the ECU using the ConS1, FCP etc.:
Valves V1 to V4 may remain closed,
Valve V5 may be opened,
Valve V6 may remain closed, and
Valve V7 may be opened.

The diluent or other fluid in the line, e.g. within channels CH1 and CH2 may be flushed out using e.g. liquid from the IV carrier fluid. If the diluent or other fluid is flushed, the pump P may be stopped by the ECU.

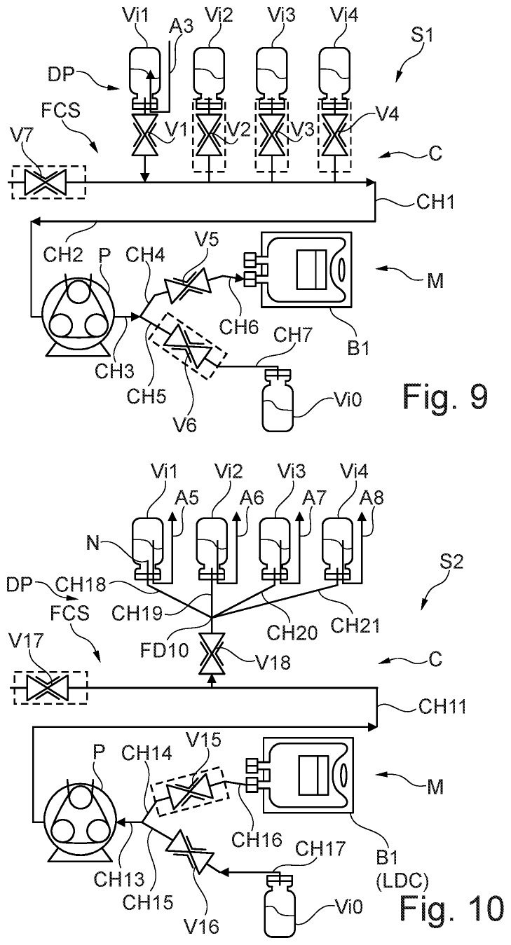

### FIG. 9 Illustrates a Step St6—Transfer of Drug Solution to IV Carrier or to Another Drug Container or Transfer Step

The following valve states may be established by the ECU using the ConS1, FCP etc.:
Valve V1 may be opened,
Valves V2 to V4 may remain closed,
Valve V5 may remain open,
Valve V6 may remain closed, and
Valve V7 may be closed.

Step St6 may follow after step St5. The pump P may be switched on by the ECU and a specific number of revolutions may be used. The rotation direction may be opposite to the previously used rotation direction of pump P. Fluid may be transferred from e.g. drug vial Vi1 (or another first drug container DC) to IV carrier or to another appropriate liquid drug container. Air may flow into the drug vials by hydrophilic vent which may be part of the consumable to vial connection which may allow air only to freely escape/enter, see arrow A3.

Step St6 may be repeated for the other drug containers DC, e.g. drug containers Vi2 to Vi4 sequentially. At the end, all valves V1 to V7 may be closed and the pump P may be switched off by the ECU. However, alternatively, pump P may not be switched off during switching of the valves V1 to V4.

At the end of the transfer step(s) St6, the pump may be switched off by the ECU and/or all valves may be closed by the ECU.

A user, e.g. HCP or non-HCP may remove and use liquid drug container LDC, e.g. for an infusion. Fluid guide system FGS may be taken out of the support portion SP/receiving space RS and may be disposed, e.g. preferably together with used drug containers DC. The auxiliary container AC may be also disposed.

The medical device M may be prepared again for the following reconstitution or other drug handling process when necessary.

### FIG. 10 Illustrates a Medical Device M Comprising a Fluid Guide System FGS Comprising a Flow Divider FD10

Flow divider FD10 may comprise a fluid distributor, e.g. comprising at least one bifurcation, see remarks in the introductory portion, feature F3.1.1.2.

A reconstitution system S2 has similar components as the system S1, e.g.:
Four drug ports DC illustrated in FIG. 10 correspond to the four drug ports DC of FIG. 4,
Again, four drug containers, e.g. vials Vi1 to Vi4 are connected mechanically and fluidically to a respective one of the drug ports DC. A different number of drug ports DP may be used as well, e.g. in the range of 1 to 50.
Channels CH11 to CH17 correspond to the channels CH1 to CH7 respectively, e.g. the channel CH17 corresponds to the channel CH7,
An auxiliary container AC is fluidically connected to the channel CH17,
A liquid drug container LDC, e.g. a plastic infusion bag may be fluidically connected to the channel CH6,
A pump P may be fluidically connected between the channels CH12 and CH13, and
Valves V15, V16 and 17 correspond to the valves V5, V6 and V7 respectively.

However, there are the following differences between system S1 and system S2:
Only one valve V18 may be used instead of the four valves V1 to V4. One side of the valve V18 may be fluidically connected with the channel CH11. The other side of the valve V18 may be fluidically connected with the flow divider FD10,
Four channels CH18 to CH21 may be fluidically connected to the flow divider FD10 on one side. The other side of the channels CH18 to CH21 may be connected with respective needles N of the four drug ports DP.

The system S2 may have similar states St1 and St2 as described above for system S1, see FIG. 4 (pre-use state) and FIG. 5 (priming state).

In step St3 the following valve configuration may be valid:

fluid channel CH, CH 10 to CH17
Valve V16 may be in an open state,
Valves V15 and V17 may be in a closed state, and
Valve V8 may be in an open state.

The step St3, e.g. simultaneous dilution/reconstitution of all vials or other drug containers DC as illustrated in FIG. 10 may correspond to e.g. step(s) St3, see FIG. 6 (filling of the first drug container, e.g. of vial Vi1) and to step St4a to 4x, see FIG. 7 (filling of the second container DC to the fourth drug container DC, e.g. of vials Vi2 to Vi4).

Thus, fluid may be pumped from a liquid source, e.g. a diluent source to all drug containers DC simultaneously and/or at the same time, e.g. to all vials Vi1 to Vi4. Thus, liquid may be pumped in parallel through all channels CH18, CH19, CH20 and CH21 in parallel, e.g. to all channels between fluid divider FD10 and the drug ports DP. In other words, when fluid is pumped to a first one of the drug containers, fluid may also be pumped to a second one (or further ones) of the drug containers.

The number of drug ports may be in the range of e.g. 2 to 100. The liquid volume may be pumped under control of the ECU by the pump P, e.g. a positive displacement pump. The fluid flow may be equally divided (e.g. split) using the flow divider FD. Air may be vented from all drug containers DC, e.g. from all drug vials Vi1 to Vi4 simultaneously by hydrophilic vents which may be part of the consumable-to-vial-connection which may allow air only to freely escape/enter, see arrows A5 to A8.

The following sequence of operation steps may be the same as mentioned above for system S1:
An optional separate reconstitution step St, e.g. supported by further measures as mentioned in this document, e.g. mechanical agitation by a user and/or by the machine, warming of the vials, pumping fluid into and out of the vials, etc. A control system may be configured accordingly, especially in order to prevent damage to the sensitive drugs D during reconstitution and/or mixing.
A step St5, i.e. flush channels CH11 and CH12 with liquid from container LDC, e.g. carrier B1, see FIG. 8,
A step St6, i.e. transfer of drug solution from drug containers DC, e.g. vials Vi1 to Vi4 to IV carrier, see FIG. 9.

Moreover, the same final steps as mentioned above may be performed also by the system S2, e.g. switching off of the pump P, etc.

In other embodiments, the drug containers may be fluidically connected in a series manner. Alternatively or additionally, a combination of parallel fluidic connection of drug containers and of series fluidic connection of drug containers

Further with reference to FIG. 10 the following may be implemented: A preferably flexible inlet tube IT (e.g. channel CH7, CH17) may be fluidically connected to the auxiliary container AC, e.g. vial Vi0 or to a flexible container, e.g. to a plastic bag. A preferably flexible outlet tube OT (e.g. channel CH6, CH16) may be fluidically connected to the liquid drug container LDC, e.g. to the flexible container B1 (bag). Hole Ho1, Ho2 may be arranged within the cover CV. Hole H1 may allow passage of the inlet tube IT from outside into the retaining space RS. Similarly, hole H2 may allow passage of the outlet tube OT from the retaining space RS to the outside.

### FIG. 11 Illustrates a Reusable Medical Device M in a State with a Closed Cover (Member) CV

The device M may be part of a system S3, e.g. a reconstitution system or other drug handling system.

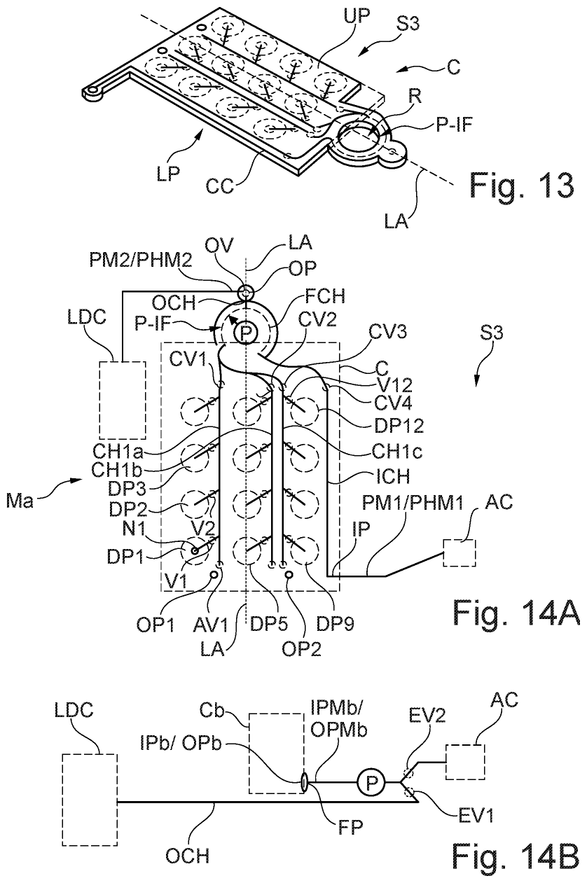

The system S3 may comprise:
The device M, and
A cartridge C as an implementation of a fluid guide system FGS as mentioned above. The cartridge C is illustrated in FIG. 12 and described in more detail below with reference to FIGS. 13 and 14.

The medical device (machine) M may comprise an outer rigid case CA. Further, the medical device M may comprise three portions P1 to P3:
A left portion P1 may comprise a fluid input portion and a user interface (UI) portion. The portion P1 may be configured to receive an auxiliary container AC, e.g. on a support portion SP1. The left portion P1 is described below in more detail.
A middle portion P2 may be a receiving portion for the cartridge C (not illustrated in FIG. 11, see FIG. 12) or for a flexible carrier B (see FIG. 17). The middle portion P2 may e.g. be covered by a cover member CV. Drug containers DDC may be coupled to drug ports DP of the cartridge C or of the carrier in a prepared state of device M. Thus, the drug containers DDC may be arranged above the middle portion P2. The middle portion P2 is described below in more detail.
A right portion P3 may be a fluid output portion and may comprise e.g. a support portion SP2 that is described in more detail below.

The left portion P1 may be configured to receive the auxiliary container AC, e.g. a plastic carrier, preferably comprising a diluent. The diluent may be WFI (water for injection). An auxiliary support portion SP1 in portion P1 of device M may allow to support the auxiliary container AC in a horizontal position, e.g. in a lying position. The shape of the support portion SP1 may be concave, e.g. in order to prevent rolling down of auxiliary container AC. Thus, the device M may be shaken slightly, e.g. in order to expedite reconstitution. A first port PM1 or a first port holding element PHM1 of the medical device M may be arranged on cover member CV on a “leg” that extends to the left portion P1 of the device M. Fluid may flow out of auxiliary container AC through the first port PM1 or the first port hold by the first port holding element PHM1 into an input port IP of the cartridge C.

An electronic control unit ECU may be arranged e.g. in the left portion P1. In an alternative embodiment, the ECU may be arranged alternatively or additionally in at least one of the other portions P2 and/or P3. The ECU may comprise a processor, a memory, a power unit, a data input unit and/or a data output unit.

The data output unit may be a movable, e.g. pivotable display unit PDU or a fixed display unit. The pivotable display unit may comprise a display unit DU, e.g. a display, a touch display, etc. Data input element(s) IE, e.g. push button(s) or a touch display may be arranged on portion P1 as well, e.g. on the display unit DU. An optional hinge H1 may allow pivoting of the pivotable display unit PDU. Alternatively, a slidable display unit DU may be used.

The middle portion P2 of the device M may comprise a support portion SP/receiving space RS for the cartridge C, see FIG. 12. The dry drug containers DDC may be coupled to the cartridge C when the cartridge C is received within the support portion SP/receiving space RS. In the embodiment, e.g. 12 vials are used as dry drug containers DDC. The 12 vials may be arranged e.g. in three columns and four rows. Alternatively, another number of dry drug containers DDC, e.g. of vials may be used. The number of dry drug containers DDC may be in the range of 1 to 50. Moreover, another number of columns and/or another number of rows may be used.

The middle portion P2 may comprise at least one output element, e.g. output elements OE1, OE2. The output elements OE1 and OE2 may be illuminated indicators, e.g. illuminated by a respective LED. The indicator OE1 may have letters, e.g. “reco” in order to signal that the reconstitution or other drug handling takes place. The indicator OE2 may have letters, e.g. “fill” in order to signal that the dry drug containers DDC and/or the liquid drug container LDC is filled. Alternatively or additionally, the at least one output element may be arranged in another portion of device M, e.g. in portion P1 and/or in portion P3.

Thus, simple visual indicators may be used to provide the user feedback of status and/or errors. However, the visual indicators may not be used if there is a screen.

A pump P may be arranged within the portion P2. Slots SL may allow dissipation of heat generated during operation of the pump P. The pump may be arranged such that an actuator of the pump P engages a recess R within the cartridge C, e.g. a circular recess. The pump P may be a peristaltic pump or pump of another type of pump.

At least one valve V, V1 may be arranged completely or partially within the portion P such that it may interact with a valve portion of the cartridge C, e.g. with a flow control portion FCP as mentioned above.

The ECU may be configured to control the pump P and the at least one valve V, V1 or valve portion V, V1. Moreover, the ECU may be configured to receive input from a user using the data input elements. Further, the ECU may be configured to signal data to the user, e.g. using display unit PDU, DU and/or optical output elements OE1, OE2 and/or acoustical output elements, etc.

Further, the middle portion P2 may comprise a second port PM2 or a second port holding element PHM2 of the medical device M. The second port PM2 or the second port holding element PHM2 may be arranged on the cover member CV (cover).

The right portion P3 may comprise the support portion SP2 that may be configured to receive liquid drug container LDC, e.g. an infusion bag. The support portion SP2 may have a concave surface in order to prevent that the liquid drug container LDC rolls down, e.g. even if the device M is shaken slightly in order to promote reconstitution of drug D within the drug containers DDC. Alternatively, the support portion SP2 may be arranged in another portion, e.g. in portion P1 and/or P2 of device M. An optional hinge H2 may be used in order to allow pivoting of support portion SP2 about an axis that extends between portions P2 and P3. Alternatively, the support portion SP2 may be fix or may be slidable, e.g. along a direction that is perpendicular to the axis mentioned above.

Further with reference to FIG. 11, a medical device M for drug D handling is provided, comprising:
A case CA,
A support portion SP, RS arranged within the case CA or on the case CA and configured to support a removable fluid guide system FGS, preferably a fluid guide system FGS according to any one of embodiments mentioned below or a fluid guide system FGS of one of the sets as mentioned below, and
At least one electronic control unit ECU.

The fluid guide system FGS may comprise at least two drug ports DP configured to be connected to a respective one of at least two drug containers DC comprising at least one drug D. The at least one electronic control unit ECU may be configured to control the operation of the medical device M during drug handling using the at least one drug D.

The fluid guide system FGS may comprise at least one flow control portion FCP configured to control flow in a fluid channel system FCF of the fluid guiding system FGS in a flow-through operation mode and in a blocking operation mode. The medical device M may comprise:
At least one flow control actuator that is configured to interact with the at least one flow control portion FCP of the removable fluid guide system FGS.

The at least one electronic control unit ECU may be configured to control the operation of the at least one flow control actuator during drug handling using the at least one drug D.

The at least one electrical control unit ECU may be configured to control the operation of the medical device M such that at least two drugs D contained in the at least two drug containers DC coupled to the at least two drug ports DP are used for the drug handling, wherein the drug handling includes reconstitution of at least one of the at least two drugs D.

The medical device M may comprise a positive displacement pump P, e.g. a peristaltic pump.

The medical device M may comprise a movable cover member CV configured to cover the fluid guide system FGS when the fluid guide system FGS is supported by the device M. The cover member CV may comprise a plurality of openings OP4. Each opening of the plurality of openings OP4 may be configured to receive a drug container DC.

Preferably, the cover CV may comprise at least one hole or at least two holes Ho1, Ho2. The hole or the holes Ho1, Ho2 may be configured to allow arrangement of a portion of an inlet tube IT and the arrangement of a portion of an outlet tube OT within the at least one hole or at least two holes Ho1, Ho2. The inlet tube IT may be configured to be fluidically connected or may be fluidically connected to an inlet port IP of the fluid guide system FGS. The outlet tube OT may be configured to be fluidically connected or may be fluidically connected to an outlet port OP of the fluid guide system FGS. Thus, e.g. two holes Ho1, Ho2 may be used. Alternatively, a common hole may be used, i.e. only one hole.

The medical device M may comprise:
At least one movable actuator element CA configured to move at least one needle N arranged within or on the fluid guide system FGS through a closing element of at least one of the at least two drug container DC.

A method for drug D handling is provided, comprising:
Using a fluid guide system FGS that comprises a fluid channel system FCS and at least two drug ports DP, each drug port being configured to receive a fluid comprising at least one drug D from at least one drug container DC,
Using a medical device M comprising a supporting portion SP, RS for the fluid guide system FGS,
Mechanically coupling the at least two drug containers DC to the at least two drug ports DP jointly during handling of the at least drug D,
Applying the fluid guide system FGS to the supporting portion SP, RS of the device M before or after coupling, and
Performing at least on drug handling step automatically using the at least one medical device M when the fluid guide system FGS is applied to the supporting portion SP, RS of the device M.

### FIG. 12 Illustrates the Reusable Medical Device M in a State with an Open Cover Member CV

Thus, the retaining space RS is visible and a cartridge C may be inserted into the retaining space RS or removed out of the retaining space RS by a user of device M. The cover member CV may cover the retaining space RS in its closed position, see FIG. 11. The cartridge C will be described in detail with reference to the next FIGS. 13 and 14 below.

In the preparation state of the device M that is illustrated in FIG. 12, the LDC support portion SP2 is still empty. Moreover, no connecting tube is coupled to port PM2 or to the second port holding element PHM2.

### FIG. 13 Illustrates a Perspective View to an Embodiment of a Consumable Fluid Guide System FGS, e.g. of a Cartridge C

The cartridge C may be usable together with the medical device M of the system S3. The cartridge C may have a cuboid main body, e.g. having a rectangular base area. The cartridge C may have a flat shape, e.g. comprising or having a height that is much smaller than its width and its length, e.g. less than 10 percent of its width. The lengths of the cartridge C may be greater than its width. The length of the cartridge C may be measured along its longitudinal axis LA. Alternatively, the cartridge C may have any other shape of the base area, e.g. square, circular, etc. According to the illustrated embodiment, one long side edge of the cartridge C may be shorter than the other long side edge, e.g. in order to save material. Alternatively, both side edges of the cartridge C may have the same lengths. A non-symmetric shape of the cartridge C may prevent errors during insertion of the cartridge C into the support portion SP/receiving space RS.

In the embodiment, the cartridge C may comprise two further deviations from the cuboid shape:
A “leg” portion on one of its shorter sides, and/or
A semicircular portion on the other of its shorts sides.

The “leg” portion may be a cuboid portion comprising or having a length that is greater than its width. The height of the “leg” portion may be the same as the height of the main body of the cartridge C. The “leg” portion may comprise the first part of the input channel ICH, see FIG. 14A and corresponding description below. Moreover, the “leg” portion may comprise the fluid input port IP which may be coupled to port PM1 or to the first port that is arranged/hold in the first port holding element PHM1 of the medical device MD. Thus, the “leg” portion may enhance design freedom for arranging port PM1 or first port holding element PHM1 of the medical device MD, e.g. within a greater distance to the drug ports DP and/or to (dry) drug containers (D) DC coupled to these drug ports DP.

The semicircular portion may comprise a base area that is more than, equal to or less than an exact semicircle or semi ring. A circular recess R may be comprised preferably partially within the semicircular portion. The other part of the circular recess R may be comprised in the main body of cartridge C. The recess R may form a receiving portion for an actuator of the pump P, e.g. for an actuator of a peristaltic pump P. The actuator of the peristaltic pump may be at least one wheel, at least two wheels or at least three wheels configured to actuate with a flexible fluid chamber portion FCH, e.g. a flexible tube portion of the fluid guide system FGS, e.g. of the cartridge C or of a flexible carrier B, see FIG. 17. The recess R and/or the fluid chamber FCH may be part of a pump-interface P-IF.

The cartridge C may be produced using injection molding or milling. Fluid control portions FCP, e.g. valve portions may be made of a flexible material, e.g. using two component molding. The same may apply to a fluid chamber FCH which is actuated by an external pump actuator in order to generate an internal fluid flow. However, other manufacturing techniques may be used as well, e.g. additive techniques, preferably 3D (three dimensional) printing. Thus, a combination of rigid molded or rigid milled parts and of flexible tube portions may be used, e.g. for the flow control portions FCP and/or for a pump interface P-IF configured to receive a mechanical force of an actuator of the pump P.

The cartridge C may comprise an outer case CC. The outer case CC may comprise:
A lower portion LP, and
An upper portion UP.

The lower portion LP may have carrier function for the channels, e.g. CH1 to CH15 of the cartridge C. The lower portion UP may be a molded part or a milled part. The lower portion LP may be made of plastic or of another appropriate material, e.g. metal. The recess R may extend through the lower portion LP.

The upper portion UP may also have a carrier function for the channels, e.g. CH1 to CH15. The upper portion UP may be a molded part or a milled part, e.g. a plastic part or a metal part. The recess R may extend through the upper portion UP.

Thus, recessed portions of the channels, e.g. CH1 to CH15 may be formed in the lower portion LP and in the upper portion UP. Alternatively, recessed portions of the channels, e.g. channels CH1 to CH15 may be formed only in the lower portion LP or only in the upper portion UP. The respective other portion may comprise only flat portions of the channel(s), e.g. e.g. channels CH1 to CH15. This may simplify production of the respective portion that comprises flat cover portions for the channels only.

A sealing element may be used between the lower portion LP and the upper portion UP, e.g. a gasket. Alternatively, a sealing technique may be used, e.g. a gluing technique and/or a heating technique, e.g. laser heating.

At least one of the lower portion LP and/or the upper portion UP may be transparent or translucent, e.g. in order to enable visual inspection of the fluid channel system FCH of the fluid guide system, e.g. before usage of the cartridge C within medical device or after usage. A user, e.g. HCP or non HCP may inspect the cartridge C.

Further with reference to FIG. 13, a fluid guide system FGS usable for drug D handling is provided, comprising:
At least one fluid port FP configured to dispense a fluid from the fluid guide system FGS,
At least two drug ports DP, each drug port DP being configured to receive a fluid comprising at least one drug D from at least one drug container DC.

Each of the at least two drug ports DC may be coupleable or coupled to at least one drug container DC such that a plurality of drug containers DC can be or is jointly mechanically coupled to the fluid guide system FGS.
A fluid channel system FCS, and
A carrier C, B configured to carry the fluid channel system FCS.

Each of the at least two drug ports DP may comprise a respective fluid transmitting portion N configured to transmit fluid coming from the respective drug port DP of the at least two drug ports DP. The fluid channel system FCS may comprise the at least one fluid port FP and the fluid transmitting portions N of the at least two drug ports DP. The fluid channel system FCS may be configured to be supported by a medical device M.

In the fluid guide system FGS at least one of the following variants a) to c) may be implemented: Variant a) A width and a length and/or a height of the fluid guide system FGS may be adapted to a width and a length and/or a height of a retaining space RS of a medical device M for retaining the fluid guide system FGS, preferably to the coverable retaining space RS that is configured to be covered by the cover CV.

Variant b) At least one hole or other recess R may be provided for the arrangement of a pump P or of at least a part of the pump P on the fluid guide system FGS, preferably a positive displacement pump, e.g. a peristaltic pump. Preferably, the pump P may be part of the medical device M that comprises a retaining space RS for retaining the fluid guide system FGS.

Variant c) An inlet port IP of the fluid guide system FGS may be configured to be connected or is connected to the flexible inlet tube IT. An outlet port OP of the fluid guide system FGS may be configured to be connected or may be connected to the flexible outlet tube OT.

Preferably, the geometrical arrangement of the inlet port IP relative to the outlet port OP may correspond (e.g. be equal) to the geometrical arrangement of at least one hole or of at least two holes Ho1, Ho2 providing access to the retaining space RS of the medical device M (e.g. machine). The retaining space RS may be configured to retain the fluid guide system FGS.

Further preferably, the at least one hole or the at least two holes Ho1, Ho2 may be configured to allow passage of a portion of the inlet tube IT and of a portion of the outlet tube OT.

The carrier C may comprise a rigid cartridge C. The rigid cartridge C may comprise a rigid case CC. The rigid case CC may comprise at least two openings OP4 configured to allow access to the at least two drug ports DP in order to couple a respective one of the plurality of drug containers D to a respective one of the at least two drug ports DP.

The carrier may comprise a flexible carrier portion FP. At least a part of the fluid channel system FCS may be formed integrally with the flexible carrier FC by connecting at least two flexible sheet materials selectively thereby forming the fluid channel system FCS.

The fluid guide system FGS may comprise at least one flow control portion FCP. At least one of the at least two drug ports DP may be or the at least two drug ports DP may be fluidically connected or connectable to the at least one fluid port FP via the at least one flow control portion FCP. The at least one flow control portion FCP may be configured to allow a fluid flow through the at least one flow control portion FCP in a flow-through operation mode of the at least one flow control portion FCP and/or to prevent a fluid flow through the at least one flow control portion FCP in a blocking operation mode of the at least one flow control portion FCP.

According to a further embodiment of the fluid guide system, the at least one drug portion may comprise at least one respective needle. The respective needle may be arranged movably relative to a case of the fluid guide system or the respective needle may be arranged within or on a flexible bag that forms the carrier of the fluid guide system.

The fluid guide system FGS may comprise at least two flow control portions FCP of the at least one flow control portion FCP. A fluid flow to each one of the at least two drug ports DP may be controllable via the operation mode of a respective one of the at least two flow control portions FCP.

The at least two drug ports DP may comprise at least four drug ports DP. The at least four drug ports DP may be arranged in a matrix Ma4 comprising at least two columns and at least two lines. The columns may be arranged perpendicular relative to the rows. Alternatively, row and columns may form another angle, e.g. in the range of 10 to 80 degrees or in the range of 20 to 70 degrees or on the range of 30 to 60 degrees.

The fluid guide system FGS may comprise at least one channel CH1a to CH1c extending along a plurality of the at least two drug ports DP. At least two auxiliary channels may connect a respective drug port DP of the at least two drug ports DP fluidically with the channel CH1a to CH1c. The respective auxiliary channel and the channel CH1a to CH1c may form an angle within the range of 10 degrees to 80 degrees, in the range of 20 degrees to 70 degrees or in the range of 30 degrees to 60 degrees.
At least one output port OP configured to be connected or connectable to a further drug container LDC, and
At least one input port IP configured to be connected or connectable to an auxiliary drug container.

The at least one output port OP and the at least one input port IP may both be fluidically connectable or connected to at least one intermediate fluid channel CH3, CH13 of the fluid guide system FGS.

Variant a): The fluid guide system FGS may comprise a pump interface unit selectively coupleable to the at least one input port IP and to the at least one output port OP.

Variant b): The fluid guide system FGS may comprise a pump selectively coupleable to the at least one input port IP and to the at least one output port OP.

### FIG. 14A Illustrates a Top View to the Fluid Guide System FGS According to FIG. 13, e.g. to the Cartridge C

In another embodiment, the cartridge C may be slightly modified, e.g. without “extension” feature (leg on one side and/or semicircular extension). Both long side surfaces of the cartridge C may have the same length in the modified embodiment.

The fluid channel system FCS of the cartridge C has the following general structure:
Drug ports DP1 to DP12 may be arranged in a matrix or in another appropriate manner. Each drug port DP1 to DP12 may comprise a needle N, N1, see e.g. FIG. 18. The needles N may be stationary or movable linearly. As already mentioned, more or less than 12 drug ports may be used per cartridge C,
Three main channels CH1a to CH1c, e.g. straight channels may be arranged parallel to each other and/or parallel to the longitudinal axis LA of the cartridge C. Alternatively, less or more than three main channels CH1a to CH1c may be used. Each main channel CH1a to CH1c may be fluidically connected to a number of drug ports DP1 to DP12, e.g. to four of the drug ports DP1 to DP12 as is described in more detail below. Each channel CH1a to CH1b may correspond to channel CH1 mentioned above, see e.g. FIGS. 4 to 10,
A fluid chamber FCH forming a pump interface P-IF, e.g. a flexible tube configured to interact with pump P (not part of the cartridge), e.g. a peristaltic pump P or another appropriate pump P,
An input channel ICH, preferably a straight channel or a channel comprising a straight main portion, e.g. extending parallel to the longitudinal axis LA,
An output channel OCH, e.g. extending parallel to the longitudinal axis LA. The output channel OCH may be comparably short if compared e.g. with the lengths of the main channels CH1a to CH1b or with length of the input channel ICH,
Valve portion V1 to V12, e.g. fluid control portions FCP, and
Channel valves CV1 to CV4 or valve portions CV1 to CV4 which may be arranged fluidically at end of the main channels CH1a to CH1b and at the end of the input channel ICH, respectively, and
An output valve OV, e.g. arranged at the outflow end of the output channel OCH.

Moreover, the following is illustrated in FIG. 14A:
The port PM1 or the first port holding element PHM1 of medical device M, e.g. enabling a fluidic and/or mechanical connection to the auxiliary container AC, e.g. the diluent,
The port PM2 or the second port holding element PHM2 of medical device M, e.g. enabling a fluidic and/or mechanical connected to the liquid drug container LDC,
Air valve portions AV1 to AV3, at the “lower” end of channels CH1a to CH1c respectively. The air valve portions AV1 to AV3 may be used for venting, e.g. during priming, see step St2 as mentioned above or during flushing, see step St5 as mentioned above, or for other purposes, and
At least one opening OP1, OP2, e.g. for venting.

The drug ports which are fluidically connected to one of the main channels CH1a to CH1c may form a column of the matrix Ma of the drug ports DP, e.g. DP1 to DP12. In the illustrated embodiment, four drug ports DP are used per channel CH1a to CH1c, e.g. in one column. The number of drug ports in one column of the matrix Ma may determine the number of lines in the matrix Ma, e.g. four lines in the embodiment. Thus, there is a 3 by 4 matrix Ma arrangement of drug ports DP and/or of (dry) drug containers (D) DC, e.g. of dry drug containers DDC. Alternatively, less than four or more than four drug ports DP may be used per column of the matrix Ma. Alternatively or additionally, less than three columns or more than three columns may be used in matrix Ma. Further, alternatively other arrangements of the drug ports may be used, e.g. circular, linear, etc.

The drug ports DP of one column may be connected to the main channels CH1a to CH1c by short auxiliary channels, e.g. straight channels. The auxiliary channels and the respective main channel CH1a to CH1c may form one side of a fishbone structure, e.g. an acute angle between the respective auxiliary channel and the main channel may be in the range of 30 to 80 degrees.

Alternatively, e.g. a perpendicular (about 90 degrees or 90 degrees) arrangement of a main channel CH1a to CH1c and the respective short auxiliary channel may be used. The auxiliary channels may comprise the respective valve (portions) V1 to V12, see e.g. valve portion V1 in the auxiliary channel that connects needle N1 of drug port DP1 and channel CH1a, valve portion V2 in the auxiliary channel that connects the needle N of drug port DP2 and channel CH1a, etc.

Thus, there is e.g. a drug port DP1 in the lower left corner. A drug port DP2 is arranged in the same column but near to fluid chamber FCH. A drug port DP3 follows, etc. on channel CH1a. On the right side of drug port DP1, a drug port DP5 may be arranged. The drug port DP5 may be connected to channel CH1b, e.g. to the second column channel CH1b. On the right side of drug port DP5, a drug port DP9 may be arranged. Drug port DP9 may be connected to channel CH1c, e.g. to the third column channel. A drug port DP12 may be arranged in the upper right corner of the matrix Ma.

The fluid chamber FCH may comprise the pump interface P-IF. The fluid chamber FCH may comprise e.g. a flexible tube configured to be pressed and released by interaction with the pump P, e.g. peristaltic pumps. The flexible tube may extend only at the location at which interaction with the pump P is possible, see e.g. arrow. Alternatively, the flexible tube may extend along at least a half of a circle or along at least three quarters of a circle as illustrated.

One end (e.g. a first end) of the fluid chamber FCH may be fluidically connected to a channel that is fluidically connected to the channel valve (portion) CV4 and/or directly to the input channel ICH.

The other end (e.g. a second end) of fluid chamber FCH may be fluidically connected to the channel valve (portions) CV1 to CV3, e.g. via only one channel or via separate channels. Alternatively, channel valve (portions) CV1 to CV3 may not be used because each drug port DP may have its own valve (portion) V1 to V12.

The output channel OCH may be fluidically connected to a middle portion of fluid chamber FCH or to another appropriate portion of the fluid chamber FCH. The actuator, e.g. EMA2, EMA4 of the pump P may actuate on a portion indicated by an arrow, i.e. on a portion that is fluidically arranged between the second end of the fluid chamber FCH and the bifurcation point on which the output channel OCH is arranged.

The cartridge C may be used to perform the methods as described above with reference to FIGS. 4 to 9 and with reference to FIG. 10, respectively. Thus, the same technical effects may apply.

In another embodiment, valve portions V1 to V12 may not be used, e.g. the drug ports DP of each column may be filled or emptied only simultaneously to each other.

The same arrangement of the fluid channel system FCS may not only be used in a rigid cartridge C but also in a corresponding flexible carrier B, see FIG. 17 and corresponding description below.

### FIG. 14B Illustrates a Variant of the Embodiment of FIG. 14A

A cartridge Cb may be slightly modified if compared with the cartridge C. The cartridge Cb may be a cartridge for which line L2 as illustrated in FIG. 4 may be valid.

There may be the following differences between cartridge C and cartridge Cb:
Cartridge Cb may not comprise a pump interface P-IF, e.g. the circular portion may be omitted.
Cartridge Cb may not comprise an output channel OCH
The input channel ICH may be connected to all channel valves (portions) CV1 to CV3. Again the channel valves CV1 to CV3 as well as channel valve CV4 may be optional because each drug port DP may comprise its own valve (portion) V1 to V12. However, again, alternatively only channel valves V1 to V3 may be used and no individual valve (portion) V1 to V12.

FIG. 14B illustrates the fluid port FP of the cartridge Cb, e.g. of the fluid guide system FGS. The fluid port FP may be a combined input/output port IPb/OPb, see also FIG. 4, Line L2. The fluid port FP may be also fluidically connected to a combined input/output port IPMb/OPMb of the medical device M, see also FIG. 4 as well as FIG. 14B.

The combined input/output port IPMb/OPMb of the medical device M may be fluidically connected to an external pump P (e.g. completely external of cartridge C), e.g. a peristaltic pump or another appropriate pump, preferably to one end of a fluid chamber FCH which is used to generate the fluid flow. The other end of the fluid chamber FCH may be fluidically connected to a bifurcation. The bifurcation may be fluidically connected with an external valve EV1 and with an external valve EV2.

The external valve EV1 may correspond to valve V5, see e.g. FIG. 4 and/or to output valve OV, see e.g. FIG. 14A. Thus, the external valve EV1 may open or block a fluid connection to the liquid drug container LDC, e.g. an infusion bag. This fluid connection may be established via the external output channel OCH.

The external valve EV2 may correspond to valve V6, see e.g. FIG. 4 and/or to “input” valve CV4, see e.g. FIG. 14A. Thus, the external valve EV2 may open or block a fluid connection to the auxiliary container AC, e.g. a flexible carrier comprising the diluent, e.g. WFI.

Alternatively, also the output channel may be at least partially or completely arranged within the cartridge Cb. Moreover, the cartridge Cb may be used to perform the same method steps St as mentioned above for cartridge C.

All variants of cartridge Cb may also be valid for a corresponding flexible carrier B, see e.g. FIG. 17.

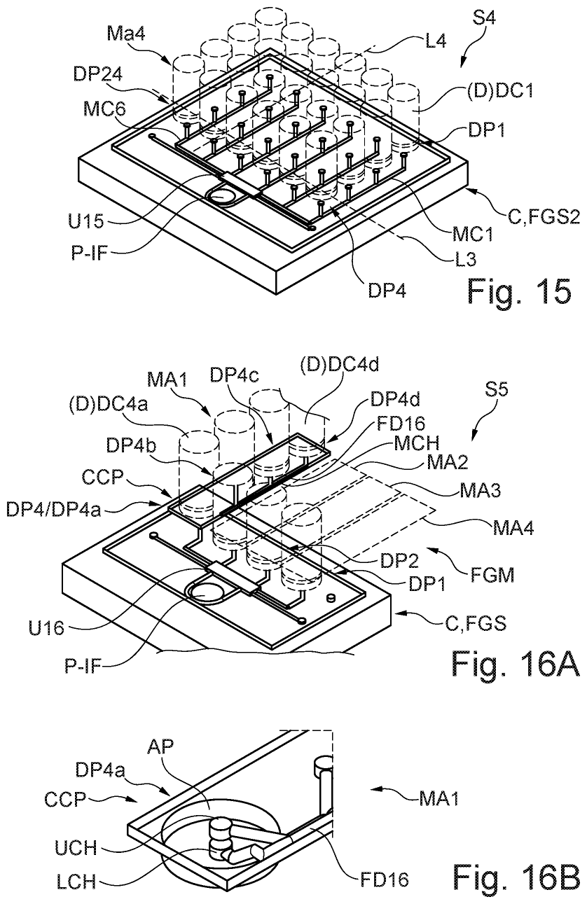

### FIG. 15 Illustrates an Embodiment of a Fluid Guide System FGS, Comprising Exactly One Drug Port (e.g. Vial Interface) for Each Drug Container (e.g. Vial).

The fluid guide system FGS may comprise a rigid cartridge C. The cartridge C may be part of a system S4. The system S4 may comprise a medical device M similar to the medical devices M of systems S0a to S3. However, the retaining space of cartridge C of system S4 may be adapted to cartridge C of system S4. The cartridge C of system S4 may comprise six columns of four drug ports DP in each column, e.g. 24 drug ports DP1 to DP24. Thus a maximum number of 24 (dry) drug containers (D) DC or alternatively of liquid drug containers DC may be coupled to these drug ports DP1 to DP24, see e.g. drug container DDC1 at drug port DP1. In other embodiments, less than 24 or more than drug ports DP/drug containers DC may be used. Moreover, less than six columns or more than six columns of drug ports DP may be used. Further, less than four lines or more than four lines of drug ports DP may be used within a matrix similar to matrix Ma4.

Variant A: Alternatively, a smaller number of drug containers may be coupled to the cartridge C or to system S4, e.g. a number of drug containers DC in the range of 1 to 24 drug containers DC or in the range of 3 to 15 drug containers (D) DC, see e.g. drug container DDC1 at drug port DP1.

Each drug port DP may comprise a needle N and a respective valve V or valve portion V. Six main channels MC1 to MC6 may have the same features as mentioned above for channels CH1a to CH1c, e.g. similar to one half of a fishbone structure of auxiliary channels and/or perpendicular arrangement between the respective main channel MC1 to MC6 and the respective auxiliary channel. The usage of one half of a “fishbone” structure may allow placing more drug ports DP within the same reference area compared to other arrangements of main channels MC1 to MC6 and of the auxiliary channels.

Main channel valves may not be used. Alternatively, main channel valves may be used, e.g. one main channel valve at the end of the main channel that is near the pump interface P-IF.

Furthermore, a unit U15 may be used to direct the fluid flows, e.g. the liquid flow generated by the pump at the pump interface P-IF. Unit U15 may comprise at least one flow distributor.

However, this drawing illustrates a high level concept and is not meant to show the fluid paths in detail. The aim of this drawings is to show that the consumable may contain a fluid connection for each vial and each vial connection may have individual valve control. The previous figures (i.e. FIG. 4) show a more thought out schematic of how the fluid control is intended to work.

However, alternatively, the individual valves V or valve portions may be switched such that any arbitrary selection of drug ports DP is possible, e.g. selection of the drug ports DP of one column (all selected drug ports are arranged at the same main channel) or of one line of the matrix arrangement Ma4.

Moreover, even an individual selection of single drug ports DP may be possible.

Variant B: A dashed line L3 illustrates a further variant B. In this variant a set of cartridges C of system S4 is sold or otherwise provided. The first cartridge may be the complete cartridge C1, e.g. comprising 24 drug ports. The second cartridge C2 may be a cartridge comprising only one line of drug ports DP, e.g. the last drug port in each column, see drug port DP4 in the first column and drug port DP24 in the sixth column and the four intermediate drug ports DP of the fourth line of the illustrated matrix Ma4. Thus, a maximum of six drug containers (D) DC may be coupled to cartridge C2. Again not all drug ports DP have to be used in order to provide flexibility for the user, e.g. with regard to the amount of dose of drug D. Thus, waste may be reduced considerably, e.g. plastic waste if a smaller cartridge C2 is used compared to cartridge C1.

According to an alternative, a further cartridge C3 of the set of cartridges may comprise two lines of drug ports or three lines of drug ports of the matrix Ma4.

Variant C: The set may comprise cartridges which comprise all drug ports of at least one complete column, see line L4, e.g. a cartridge C comprising four drug ports of the last column, e.g. including drug port DP24. Alternatively, only drug ports of the second column, etc. may be provided by the respective cartridge.

Further alternatively, drug ports of two, three, etc. adjacent columns may be provided.

The cartridge C1 may be part of this set of variant C as it comprises all drug ports DP of all six columns. However, intermediate columns may be included additionally to the last column and/or instead of the last column.

Variant D: Combination of arrangements which comprise drug ports DP of at least one complete column and of at least one complete row, e.g. similar to the arrangement as illustrated in FIG. 16 but without the modular function, e.g. an integrally formed cartridge. Again cartridge C of system S4 may be part of this set.

A combination of cartridges of any variant A to D is of course possible within one set of cartridges C.

The number of SKUs (stock keeping unit) may be reduced by selecting only the most relevant configurations of drug ports DP, e.g. a number of configurations within the set in the range of e.g. 2 to 10 or 3 to 5 configurations.

The same may be valid for a set of flexible carriers B, see FIG. 17.

Thus, the concept described in the flow diagrams, see e.g. FIGS. 4 to 10 may assume a consumable with multiple vial interfaces (drug ports DP) and e.g. a respective valve V or a valve portion to open/close access to each vial.

There are alternatives to this assumption which would follow the same flow steps St, but which would require fewer vial access interfaces and fewer valves, see e.g. variants A to D and variants illustrated in FIG. 16.

The set of fluid guide systems FGS, FGS2 may comprise at least one fluid guide system FGS according to one of the embodiments mentioned above, e.g. cartridge C (C1) of system S4 (or a corresponding carrier B) and at least one of the following:
At least one further fluid guide system FGS2, e.g. another cartridge C (e.g. cartridge C2, C3, etc.) or a carrier B, wherein the further fluid guide system FGS2 may comprise at least some of the features as the fluid guide system FGS, e.g. a basis version of the cartridge C (system S4), C1 or of a corresponding carrier B.

The further fluid guide system FGS2 may comprise compared to the fluid guide system FGS (basis version) at least one, several of or all of:
A different fluid channel system FCS, and/or
A different number of fluid control portions FCP, and/or
A different number of drug ports DP, and/or
A different size, e.g. maximum length and/or a maximum width, and/or
A different outer shape.

However, all types of the fluid guide systems FGS, FGS2 of the same set may be configured to be received within or on the same support portion SP/receiving space RS of the same medical device M. This may be reached by e.g.:
The same pump interface P-IF, and/or
The same height of the fluid guide systems FGS, FGS2 of the set, and/or
At least one common width and/or lengths of the fluid guide systems FGS, FGS2 of the set, and/or
A general common portion of all fluid guide systems FGS, FGS2 of the set, etc.

### FIG. 16A Illustrates an Alternative Embodiment of a Fluid Guide System C, FGM Comprising (Exactly) One Vial Interface (or Another Drug Container DC) for Each Vial (or Other Drug Container DC)

At least one module MA1 to MA 4, e.g. a modular flow divider may be used to divide the flow e.g. equally between several vials (or other drug containers) of a module MA1 to MA4 at once, i.e. simultaneously.

The modules MA1 to MA4 may have the same number of drug ports DP, e.g. four drug ports in the embodiment. Not all drug ports may be used to connect drug containers DC in an application scenario. Alternatively, different types of modules MA may be used with a different number of drug ports between different types of modules MA, e.g. one module having three drug ports DP and one module having four drug ports DP. Again, not all drug ports of the different types of the modules may be used in an application scenario. Thus, there is an increased flexibility for the user. However, the number of module types may be reduced for reasons of stock keeping.

Not all of the modules MA1 to MA4 have to be used in the respective application case. Thus, only one of the modules MA1 to MA4 may be used, two modules, three modules or all four modules may be used. In another embodiment, the number of drug ports on the cartridge C (or carrier B) may be less than four drug ports DP or more than four drug ports DP.

The fluid guide system C, FGM may be a component of a system S5. The system S5 may also comprise a medical device M as described above for the systems S0a to S4.

Each drug port DP1 to DP4 may comprise a respective needle N, see e.g. FIG. 18. Drug ports DP1 to DP4 may comprise corresponding valve portions or valves, e.g. arranged in the fluid flow to or from the respective drug port DP1 to DP4. The drug ports DP1 to DP4 may be coupled to respective drug containers DC, e.g. to vials, see e.g. drug ports DP1, DP2 and DP3 in FIG. 16A. In another embodiment all four drug ports DP1 to DP4 may be coupled directly to drug containers Dc, e.g. vials. However, in the embodiment illustrated in FIG. 16A a module MA1 is coupled to drug port DP4. Module MA1 may couple four further drug containers to the fluid guide system FGS, C indirectly, e.g. via a common coupling port CCP. Thus, seven drug containers (D) DC may be coupled at most to the fluid guide system FGS, C in the embodiment of FIG. 16A.

Module MA1 may comprise four drug ports DP4a to DP4d. Alternatively, less than for drug ports DP or more than four drug ports DP may be used per module. The same may apply to other modules MA2 to MA4, which may be optionally used. Dry drug containers DDC4b to DDC4d, e.g. vials may be coupled to the drug ports DP4a to DP4d.

Each module may comprise a main channel MCH which may have the function of a flow divider FD16. Details of the flow divider FD16 are described below, see FIG. 16B and corresponding description. There may be no valves or valve portion on or within a module MA1 to MA4. Thus, the modules MA1 to MA4 may be simple modules.

Thus, each drug port DP1 to DP4 may be used to couple a module MA1 to MA4 to the fluid guide system FGS, C, B. The lower part of the first port of a module is the common coupling port CCP as it is used to couple all drug ports of a respective module to the selected drug port to which the module is coupled. The modules MA1 to MA4 may be fluid guide modules FGM.

The fluid guide system FGS, e.g. cartridge C or carrier B may comprise a pump interface P-IF. A unit U16 may be used to direct the fluid flows on the “basic” fluid guide system FGS, C, B. Unit U16 may comprise at least one flow distributor. The remarks mentioned above with regard to unit U15 apply also to unit U16.

As already mentioned, alternatively, two or three or more modules, e.g. modular adapters may be used. Simultaneous filling and/or or removal (extraction) of fluid from drug ports DP/drug containers (D) DC of one modular adapter or module MA1 to MA4 may be performed.

Thus, the main consumable FGS, C may contain a drug port, e.g. a vial interface to each drug container, e.g. vial. However, the modules MA1 to MA4 may not comprise valves or valve portions. In sum, there may be fewer valves than the number of vial interfaces. A flow divider, e.g. FD16 may be used to split the flow, e.g. equally between the drug containers DC, e.g. vials of each module, e.g. MA1 to MA4. This may allow multiple drug containers, e.g. vials to be filled/extracted in parallel, e.g. simultaneously.

Similarly, modules based on plastic carriers B may be used.

A set of fluid guide systems FGS, FGM may comprise at least one fluid guide system FGS according to any one of the embodiments mentioned above and at least one of the following:
At least one fluid guide module FGM, e.g. a module MA1 to MA4 which may be configured to be coupled to the at least one drug port DP via a common coupling port CCP.

The at least one fluid guide module FGM may comprise at least two drug ports DP, e.g. DP4a and DP4b. The at least two further drug ports DP may be fluidically coupled or coupleable to the common coupling port CCP of the at least one modular fluid guide module FGM. Preferably, the modular fluid guide module FGM may be free of a flow control portion having at least two operation modes.

FIG. 16B illustrates a detail of the module MA1 of the embodiment as illustrated in FIG. 16A. A cylindrical adapter portion AP may be used to define the coupling portion which is configured similar to the coupling portion of a drug container, e.g. of a vial. The flow divider FD16 may comprise the following parts within the adapter portion AP:
A lower channel LCH configured to be part of the common coupling port CCP, and/or
An upper channel UCH configured to be part of the first drug port DP4a of the module, e.g. MA1.

The lower channel LCH and the upper channel UCH may be fluidically connected to the main channel MCH of the fluid divider FD16.

Thus, the common coupling port CCP of a modular adapter (extension, module MA1 to MA4) may be designed to mimic a drug container closure, e.g. a vial closure for interface with the main consumable, e.g. with cartridge C, B, etc.

The electronic control unit ECU may change the control if a module, e.g. MA1 is identified, e.g. manually by a user or detected automatically by the medical device M. Thus, the pumping power, e.g. revolutions per minute may be increased for filling the drug containers of a module or for pumping fluid out of the drug containers of a module.

### FIG. 17 Illustrates a Plastic Carrier B Fluid Guide System FGS, B

The fluid guide system FGS, B may be part of a system S6. The system S6 may comprise a medical device M as mentioned above. The fluid guide system FGS may be based on a plastic carrier B. Thus, the plastic carrier B may form a carrier that carries the channels of the fluid guide system FGS. Moreover, the channels of the fluid guide system FGS, B may be formed by connecting two opposite side faces of the carrier B, e.g. a plastic carrier. Other appropriate flexible materials may be used as well.

The carrier B may comprise essentially the same or similar components as the cartridge C, see FIG. 14A and corresponding description. However, the fluid channel system FCS of the carrier B may be slightly modified if compared with the fluid channel system FCS of the cartridge C, e.g. there may be no lateral “extension” feature (leg on one side), both long side edges may have the same length, etc. However, according to an alternative embodiment, these features may also be implemented in the flexible carrier B.

Thus, the carrier B may comprise similar parts as illustrated in FIG. 14A, e.g.:
A pump interface P-IF configured to interact with a pump actuator,
Main channels CH1a, CH1b and CH1c,
An input port IP configured to be fluidically connected to a port PM1 of the medical device M or to a port arranged in the first port holding element PHM1 of medical device M,
An output port IP configured to be fluidically connected to a port PM2 of the medical device M or to a second port hold in the second port holding element PHM2 of medical device M,
Drug ports DP1 to DP12,
A fluid chamber FCH,
An input channel ICH,
An output channel OCH,
Valve portions V1 to V12,
Air valve portions AV1 to AV3,
Opening(s) OP1 and/or OP2, e.g. for venting and/or comprising an air filter,
Channel valves CV1 to CV4,
An output valve OV,
Needles N, N1, etc.,
etc.

Heat sealing may be used or another connection technique in order to produce the channels and/or valves of the carrier B. Heat sealing may be performed along the lines illustrated in FIG. 17 or even in greater areas between these lines which are not occupied by channels, e.g. CH1a, CH1b, Ch1c, etc. and/or valves V1, to V12, CV1 to CV4, etc., see e.g. hatched area HA. Sealing may be performed simultaneously or sequentially.

According to an alternative, also the variant with an external pump P may be applied to carrier B, see FIG. 14B and corresponding description above.

The needles N may already be pierced to the upper side of the plastic carrier in a pre-use condition. However, usage of a separate outer plastic carrier or of caps for the needles may be an option. The caps may be removed by a user before carrier B is inserted into the support portion SP/receiving space RS of the medical device M. The needles N may be attached to needle holder members, e.g. to discs in the example. However, other shapes may be used as well, e.g. square, rectangle, ellipse. The respective needle N may have only one inlet or at least two inlets, e.g. depending on whether an air vent is integrated or not.

It may be desirable to avoid contact with the sterile areas of the consumable, e.g. such as the needles during handling to avoid contamination. This could be achieved by having a cap removed by the user. Alternatively, the needles N may be recessed in their pre-connection state and a mechanical interface may be used with the reusable to actuate them during assembly, see e.g. FIG. 18. Alternatively or additionally, a cover may be used over the needle pre-use that may be actuated during vial insertion, e.g. pierced through by the needle N.

In order to avoid unnecessary repetition, the connection of the features of carrier B is not described in detail. Reference is made instead to the connections of the features of cartridge C as mentioned above, see FIGS. 14A and/or 14B and corresponding description. The same applies to modifications of carrier B which may be the same as the modifications mentioned above for the cartridge(s) C.

The carrier B may be used for performing all methods mentioned above, see e.g. FIGS. 4 to 9 and FIG. 10 as well as corresponding parts of the description.

### FIG. 18 Illustrates an Embodiment of a Flow Control Portion FCP, e.g. a Pinch Valve Portion PVP and of a Drug Port DP, e.g. of a Vial Port

The drug port DP and the pinch valve portion PVP may be part of a fluid guide system FGS, e.g. of a cartridge C, see e.g. case CC of the cartridge C. The fluid guide system FGS may be part of a system S7. The system S7 may comprise a medical device M. Some parts of the medical device M are also illustrated in FIG. 18, e.g. cover member CV, etc.

The drug container DC may be e.g. a vial. The drug container DC may comprise a drug D, e.g. a lyophilized drug or a drug of fluid drug formulation. The drug port DP may be configured to provide engagement with the drug container, e.g. the vial. Thus, vial engagement clips VC (or other hooks) may be used to provide a non-detachable connection between the drug container DC and the drug port DP. Other connections may be used as well, e.g. a screw threaded outer part of the drug container DC engaging at least one inner screw thread of the drug port DC. During operation of the medical device M, the drug container DC may be stationary relative to the cartridge C and relative to the medical device M.

The cover unit/member CV (e.g. lid) may be configured to react a valve and/or actuator force, e.g. together with the upper side of the case CC of cartridge C. Thus, e.g. a pinch valve portion PVP may be pressed by a pinch valve actuator PVA. The pinch valve actuator PVA may be arranged externally to cartridge C within device M. The pinch valve actuator PVA may comprise e.g. a pin, preferably a pin with a conical end. A drive unit may move the pin upwards. The drive unit may comprise a linear actuator, a further cam mechanism, a coil of a solenoid, etc. The pin may be slidable within a guide unit/member.

An opening OP3 within the bottom wall of the case CC of the cartridge C may allow pinch valve actuator PVA to enter into the cartridge C. A conical indentation may be provided opposite to opening OP3 within the upper portion of the case CC, e.g. at the inside thereof in order to ease pressing of the pinch valve portion PVP of the tubing.

An opening OP4 within a top wall of the case CC of the cartridge C may allow insertion of the drug container DC and/or may be used by elements of the drug port P for fastening/attaching the drug container DC, see e.g. vial engagement clips VC extending through the opening OP4.

In the embodiment, a movable needle N (needle holding unit) is used. A cam actuator CA may be used to insert the needle N of the drug port P into and through a closure portion of the drug container DC, e.g. of the vial. The cam actuator CA may comprise an eccentric disc, e.g. with a rotation axis arranged outside of the geometrical center of the disc. The disc may be circular, elliptical, etc. The needle N may be fluidically coupled to the flexible tube that also comprises the pinch valve portion PVP.

Alternatively, a rigid channel system may be used, e.g. comprising a diaphragm valve portion and a flexible tube only to allow movement of the needle N. However, a movable needle may also be implemented using a rigid channel, e.g. comprising a telescopic portion or another appropriate portion that allows movement of the needle N and that provides a fluidical connection to the needle N.

Alternatively a stationary needle N may be used, e.g. needle insertion into a closure of the drug container DC may be done during screwing in of the drug container DC into the drug port DP, during insertion of the drug container DC into the drug port DP, etc. A flexible tube or a rigid tube may be used to connect the needle N fluidically to the fluid guide system FGS of the cartridge C.

An optional vial presence sensor VPS may be used, e.g. a mechanical contact sensor or an electronic distance sensor, e.g. an US (ultrasonic) sensor. Alternatively, the user may be asked by the machine to confirm that the vial or another drug container is in place.

Optionally, corresponding sensors may be used for the detection of presence of the auxiliary container AC and/or of the liquid drug container LDD or of other appropriate containers of device M.

The flow control portion FCP may comprise a flexible tube portion configured to be pressed by an external actuator, e.g. by an external valve actuator, see e.g. pinch valve actuator PVA.

The at least one drug portion DP may comprise at least one respective needle N. Preferably, the respective needle N may be arranged movably relative to a case CC of a cartridge C. Alternatively, the respective needle N may be arranged stationary relative to a case CC of a cartridge C. Both alternatives may also be valid for a flexible carrier B of the fluid guide system FGS.

The medical device M may comprise at least one movable actuator element configured to move at least one needle N within the cartridge C through a closing element of the at least one drug container DDC. The movable actuator may be preferably a cam element CA or a linear actuator element.

A list of features, grouped in sublevels and containing variations and alternatives is presented in the introductory part. These features and feature groups FG may be combined with any one of the embodiments of the second part of the description, e.g. with the embodiments illustrated in the figures.

Although embodiments of the present disclosure and their advantages have been described in detail, it should be understood that various changes, substitutions and alterations can be made therein without departing from the spirit and scope of the disclosure as defined by the appended claims. For example, it will be readily understood by those skilled in the art that many of the features, functions, processes and methods described herein may be varied while remaining within the scope of the present disclosure. Moreover, the scope of the present application is not intended to be limited to the particular embodiments of the system, process, manufacture, method or steps described in the present disclosure. As one of ordinary skill in the art will readily appreciate from the disclosure of the present disclosure, systems, processes, manufacture, methods or steps presently existing or to be developed later that perform substantially the same function or achieve substantially the same result as the corresponding embodiments described herein may be utilized according to the present disclosure. Accordingly, the appended claims are intended to include within their scope such systems, processes, methods or steps. The embodiments mentioned in the first part of the description may be combined with each other. The embodiments of the description of figures may also be combined with each other. Further, it is possible to combine embodiments mentioned in the first part of the description with examples of the second part of the description which relates to FIGS. 1 to 18.

1. A fluid guide system configured to interact with a medical device for performing a reconstitution of at least one lyophilized drug contained within a plurality of drug containers by transferring a diluent into the plurality of drug containers in order to reconstitute the at least one lyophilized drug to at least one reconstituted drug and transferring a fluid comprising the diluent and the at least one reconstituted drug out of the plurality of drug containers, the fluid guide system comprising:
at least one fluid port configured to dispense the fluid comprising the diluent and the at least one reconstituted drug from the fluid guide system,
at least two drug ports, wherein each of the at least two drug ports is coupleable or coupled to a respective drug container of the plurality of drug containers such that the respective drug container can be or is directly mechanically coupled to a respective drug port of the at least two drug ports, and wherein each of the at least two drug ports comprises a fluid transmitting portion configured to transmit the fluid from the respective drug container,
a fluid channel system comprising at least two different fluid paths including a first fluid path configured to transfer the diluent to the respective drug container and a second fluid path configured to transfer the fluid comprising the diluent and the at least one reconstituted drug out of the respective drug container,
at least one flow control portion comprising at least one valve portion that controls which of the at least two different fluid paths is active, wherein the at least one valve portion is configured to be actuated by at least one valve actuator of the medical device,
a carrier configured to carry the fluid channel system; and
at least one pump or at least one pump interface;
wherein each of the plurality of drug containers are vials; and
wherein the first fluid path ends on the at least two drug ports and the second fluid path starts on the at least two drug ports.

2. The fluid guide system according to claim 1, wherein at least one of a), b), or c) is implemented:
a) (i) a width and a length and/or a height of the fluid guide system are adapted to a width and a length and/or a height of a retaining space of the medical device for retaining the fluid guide system,
b) (i) at least one hole or other recess for arranging a pump or at least a part of the pump on the fluid guide system, and
(ii) wherein the pump is part of the medical device that comprises a retaining space for retaining the fluid guide system, or
c) (i) an inlet port of the fluid guide system is configured to be connected or is connected to a flexible inlet tube, and
(ii) an outlet port of the fluid guide system is configured to be connected or is connected to a flexible outlet tube, and
(iii) wherein a geometrical arrangement of the inlet port relative to the outlet port corresponds to a geometrical arrangement of at least one hole or of at least two holes providing access to a retaining space of the medical device, and
(iv) wherein the at least one hole or the at least two holes are configured to allow passage of a portion of the flexible inlet tube and of a portion of the flexible outlet tube.

3. The fluid guide system according to claim 2, wherein the carrier comprises a rigid cartridge,
wherein the rigid cartridge comprises a rigid case, and
wherein the rigid case comprises at least two openings configured to allow access to the at least two drug ports in order to couple the respective drug container to a respective one of the at least two drug ports.

4. The fluid guide system according to claim 2, wherein the carrier comprises a flexible carrier portion,
wherein at least a part of the fluid channel system is formed integrally with the flexible carrier portion by connecting at least two flexible sheet materials selectively thereby forming the fluid channel system.

5. The fluid guide system according to claim 1, wherein the at least two drug ports are fluidically connected or connectable to the at least one fluid port via the at least one flow control portion, and
wherein the at least one flow control portion is configured to allow a fluid flow through the at least one flow control portion in a flow-through operation mode of the at least one flow control portion and to prevent the fluid flow through the at least one flow control portion in a blocking operation mode of the at least one flow control portion.

6. The fluid guide system according to claim 1, wherein each of the at least two drug ports comprises at least one respective needle, and
wherein a) the at least one respective needle is arranged movably relative to a case of the fluid guide system, or b) the at least one respective needle is arranged within or on a flexible bag that forms the carrier of the fluid guide system.

7. The fluid guide system according to claim 1, wherein the at least two drug ports comprise at least four drug ports,
wherein the at least four drug ports are arranged in a matrix comprising at least two columns and at least two lines.

8. The fluid guide system according to claim 1, comprising at least one channel extending along a plurality of the at least two drug ports, and
at least two auxiliary channels connecting a respective drug port of the at least two drug ports fluidically with the at least one channel,
wherein the at least two auxiliary channels and the at least one channel form an angle a range of 30 degrees to 60 degrees.

9. The fluid guide system according to claim 1, comprising:
at least one output port configured to be connected or connectable to a further drug container, wherein the further drug container is an infusion bag, and
at least one input port configured to be connected or connectable to an auxiliary drug container,
wherein the at least one output port and the at least one input port are both fluidically connectable or connected to at least one intermediate fluid channel of the fluid guide system, and
wherein a) the fluid guide system comprises a pump interface unit selectively coupleable to the at least one input port and to the at least one output port, or b) the fluid guide system comprises a pump selectively coupleable to the at least one input port and to the at least one output port.

10. (canceled)

11. A medical device for drug handling, comprising:
a case,
a support portion arranged within the case or on the case and configured to support a removable fluid guide system,
at least one electronic control unit, and
at least one valve actuator;
wherein the removable fluid guide system comprises:
at least one fluid port configured to dispense a fluid comprising a diluent and at least one reconstituted drug based on at least one lyophilized drug from the removable fluid guide system;
at least two drug ports configured to be directly mechanically connected to a respective one of a plurality of drug containers comprising the at least one lyophilized drug, and wherein each of the at least two drug ports comprises a fluid transmitting portion configured to transmit the fluid from the respective drug container;
a fluid channel system comprising at least two different fluid paths including a first fluid path for transferring the diluent to the respective drug container and a second fluid path for transferring the fluid comprising the diluent and the at least one reconstituted drug out of the respective drug container, wherein the first fluid path ends on the at least two drug ports and wherein the second fluid path starts on the at least two drug ports;
at least one flow control portion comprising at least one valve portion that controls which of the at least two different fluid paths is active, wherein the at least one valve portion is configured to be actuated by the at least one valve actuator of the medical device;
a carrier configured to carry the fluid channel system; and
at least one pump or at least one pump interface;
wherein the drug containers of the plurality of drug containers are vials comprising the at least one lyophilized drug;
wherein the at least one electronic control unit is configured to control an operation of the medical device during the drug handling using the at least one lyophilized drug,
wherein the at least one electronic control unit is configured to select an appropriate program for reconstitution steps of the at least one lyophilized drug within the respective drug container by transferring a diluent to the respective drug container.

12. The medical device according to claim 11, wherein the removable fluid guide system comprises at least one flow control portion configured to control flow in a fluid channel system of the removable fluid guide system in a flow-through operation mode and in a blocking operation mode, and wherein the medical device further comprises:
at least one flow control actuator that is configured to interact with the at least one flow control portion of the removable fluid guide system, and
wherein the at least one electronic control unit is further configured to control an operation of the at least one flow control actuator during the drug handling using the at least one lyophilized drug.

13. The medical device according to claim 11,
wherein the at least one electrical control unit is configured to control the operation of the medical device such that at least two lyophilized drugs of the at least one lyophilized drug contained in the plurality of drug containers coupled to the at least two drug ports are used for the drug handling, and
wherein the drug handling includes reconstitution of at least one of the at least two lyophilized drugs.

14. The medical device according to claim 11, comprising a positive displacement pump.

15. The medical device according to claim 11, comprising a movable cover member configured to cover the removable fluid guide system when the removable fluid guide system is supported by the medical device, wherein the movable cover member comprises a plurality of openings and wherein each opening of the plurality of openings is configured to receive a respective drug container of the plurality of drug containers,
wherein the movable cover member comprises at least one hole or at least two holes configured to allow an arrangement of a portion of an inlet tube and an arrangement of a portion of an outlet tube within the at least one hole or at least two holes,
wherein the inlet tube is configured to be fluidically connected or is fluidically connected to an inlet port of the removable fluid guide system, and
wherein the outlet tube is configured to be fluidically connected or is fluidically connected to an outlet port of the removable fluid guide system.

16. The medical device according to claim 11, comprising:
at least one automatically movable actuator element configured to move at least one needle arranged within or on the removable fluid guide system through a closing element of the respective drug container.

17. A method for drug handling, comprising:
performing at least one drug handling step automatically using a medical device according to claim 11 when a removable fluid guide system is applied to a support portion of the medical device.

18. (canceled)

19. A computer-implemented method performed by the at least one electronic control unit of the medical device according to claim 11, comprising:
sensing or prompting a user to confirm a coupling of the plurality of drug containers to a respective one of the at least two drug ports of the removable fluid guide system,
wherein the least two drug ports are configured to receive the at least one reconstituted drug from the respective drug container.

20. The computer-implemented method according to claim 19, further comprising at least one of:
controlling a pump drive unit that is configured to drive a pump, wherein at least a part of the pump is implemented on the removable fluid guide system or wherein the pump is configured to generate a pumping force for a fluid flow within the removable fluid guide system,
prompting the user to begin a drug handling process,
performing a calculation to calculate a dose of the at least one lyophilized or reconstituted drug,
providing alerts and/or guidance to the user, or
sensing a presence of at least one further container.

21. The fluid guide system according to claim 2, wherein c) is implemented and wherein the at least one hole or the at least two holes are arranged within a movable cover of the retaining space.

22. The fluid guide system according to claim 4, wherein heat sealing has been performed to produce channels of the flexible carrier portion.

23. The fluid guide system according to claim 5, wherein the flow control portion comprises a flexible tubing portion which is configured to perform a pinch valve function when interacting with an actuator pin of the medical device,
wherein the actuator pin is external to the fluid guide system, and
wherein the flexible tubing portion is arranged at a valve actuator opening of a case of the fluid guide system which is configured to allow the actuator pin to pinch the flexible tubing portion,
wherein the carrier comprises a rigid cartridge;
wherein the rigid cartridge comprises a rigid case; and
wherein the rigid case comprises the fluid channel system, the at least one flow control portion, and the valve actuator opening.

24. The fluid guide system according to claim 6, wherein the at least one respective needle is configured to be actuated by a cam actuator of the medical device,
wherein at least one of the at least two drug ports comprises a drug container opening for the respective drug container;
wherein the at least one respective needle is arranged within the case of the fluid guide system and at a drug container opening of the case of the fluid guide system;
wherein a cam actuator opening is arranged on the case of the fluid guide system opposite to the drug container opening; and
wherein the cam actuator opening is configured to allow an arrangement of the cam actuator of the medical device within the cam actuator opening and to allow the cam actuator to move the at least one respective needle.

25. (canceled)

26. (canceled)

27. The fluid guide system according to claim 9, wherein the further drug container is configured to receive the fluid and to store the fluid intermediately for later use after decoupling of the further drug container from the at least one output port.

28. The medical device according to claim 12, wherein the at least one flow control portion comprises a flexible tubing portion which is configured to perform a pinch valve function when interacting with an actuator pin of the medical device,
wherein the actuator pin is external to the removable fluid guide system;
wherein the flexible tubing portion is arranged at a valve actuator opening of a case of the fluid guide system which is configured to allow the actuator pin to pinch the flexible tubing portion;
wherein the carrier comprises a rigid cartridge;
wherein the rigid cartridge comprises a rigid case; and
wherein the rigid case comprises the fluid channel system, the at least one flow control portion, and the valve actuator opening.

29. The fluid guide system according to claim 1, comprising at least one vent which is part of a connection between the fluid guide system and the plurality of drug containers and which allows air only to freely escape and/or enter.

30. The method according to claim 17, further comprising:
coupling the plurality of drug containers to the removable fluid guide system;
transferring the diluent to at least one drug container of the plurality of drug containers via the first fluid path;
actuating the at least one valve portion by the at least one valve actuator in order to activate the second fluid path in order to reconstitute the at least one lyophilized drug to the at least one reconstituted drug;
dispensing a fluid comprising the diluent and the at least one reconstituted drug from the removable fluid guide system via the second fluid path.

31. The fluid guide system according to claim 1, wherein the fluid guide system comprises at least one peristaltic pump, at least one diaphragm pump, at least one peristaltic pump interface, or at least one diaphragm pump interface.

32. The medical device according to claim 11, wherein the at least one valve actuator is connected to an electronic interface of the electronic control unit.

33. The medical device according to claim 11, wherein the medical device is configured such that multiple of the plurality of drug containers can be simultaneously or sequentially connected to a single diluent source via the first fluid path.

34. The medical device according to claim 16, wherein the at least one respective needle is configured to be actuated by a cam actuator of the medical device;
wherein at least one of the at least two drug ports comprises a drug container opening for the respective drug container;
wherein the at least one respective needle is arranged within the case of the removable fluid guide system and at the drug container opening of the case of the removable fluid guide system;
wherein a cam actuator opening is arranged on the case of the removable fluid guide system opposite to the drug container opening;
wherein the cam actuator opening is configured to allow an arrangement of the cam actuator of the medical device within the cam actuator opening and to allow the cam actuator to move the at least one respective needle; and
wherein the cam actuator is the automatically movable actuator element.
